(19) United States
(12) Patent Application Publication
(10) Pub. No.: US 2022/0127609 A1
(43) Pub. Date: Apr. 28, 2022

(54) ANTISENSE OLIGONUCLEOTIDES FOR NUCLEIC ACID EDITING

(71) Applicant: ProQR Therapeutics II B.V., Leiden (NL)

(72) Inventors: Julien Auguste Germain Boudet, Leiden (NL); Lenka Van Sint Fiet, Leiden (NL)

(21) Appl. No.: 17/429,796

(22) PCT Filed: Feb. 10, 2020
(86) PCT No.: PCT/EP2020/053283

§ 371 Date: Aug. 10, 2021

(30) Foreign Application Priority Data
Feb. 11, 2019 (GB) 1901873.8

(51) Int. Cl. C12N 15/113; A61K 31/7088

(52) U.S. Cl. CPC C12N 15/113; A61K 31/7088; C12N 2310/11; C12N 2310/312; C12N 2310/344; C12N 2310/313; C12N 2310/346; C12N 2310/321; C12N 2310/315

(57) ABSTRACT

The invention relates to editing oligonucleotides (EONs) for binding to a target nucleic acid and recruiting an enzyme with nucleotide deamination activity to edit the target nucleic acid. The EONs carry phosphonoacetate internucleotide linkage modifications and/or unlocked nucleic acid (UNA) ribose modifications at specified positions and do not carry such modifications on positions that would lower nucleic acid editing efficiency. The selection of positions that should or should not carry a modification is based on computational modelling that revealed incompatibilities of the modifications with the enzyme with nucleotide deamination activity.

### TECHNICAL FIELD

The invention relates to the field of medicine. In particular, it relates to the field of nucleic acid editing, whereby a nucleic acid molecule in a cell is targeted by an antisense oligonucleotide to specifically change a target nucleotide, including the correction of a mutation in the nucleic acid sequence by an enzyme having deaminase activity. More specifically, the invention relates to antisense oligonucleotides that are chemically modified at preferred positions in such a particular specific manner that it increases their RNA editing efficiency. The chemical modifications include use of phosphonoacetate internucleotide linkers and/or unlocked nucleic acid ribose modifications.

### BACKGROUND

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are adenosine (A) to inosine (I) conversions and cytidine (C) to uridine (U) conversions, which occur through enzymes called adenosine deaminase and cytidine deaminase, respectively. The most extensively studied RNA editing system is the adenosine deaminase enzyme.

Adenosine deaminase is a multi-domain protein, comprising a catalytic domain, and two to three double-stranded RNA recognition domains, depending on the enzyme in question. Each recognition domain recognizes a specific double stranded RNA (dsRNA) sequence and/or conformation. The catalytic domain does also play a role in recognizing and binding a part of the dsRNA helix, although the key function of the catalytic domain is to convert an adenosine (A) into inosine (I) in a nearby, more or less predefined, position in the target RNA, by deamination of the nucleobase. Inosine is read as guanosine by the translational machinery of the cell, meaning that, if an edited adenosine is in a coding region of an mRNA or pre-mRNA, it can recode the protein sequence. A to I conversions may also occur in 5′ non-coding sequences of a target mRNA, creating new translational start sites upstream of the original start site, which gives rise to N-terminally extended proteins, or in the 3′ UTR or other non-coding parts of the transcript, which may affect the processing and/or stability of the RNA. In addition, A to I conversions may take place in splice elements in introns or exons in pre-mRNAs, thereby altering the pattern of splicing. As a result thereof, exons may be included or skipped. The adenosine deaminases are part of a family of enzymes known as Adenosine Deaminases acting on RNA (ADAR), including human deaminases hADAR1 and hADAR2, as well as hADAR3 for which no deaminase activity has been shown yet.

The use of oligonucleotides to edit a target RNA applying adenosine deaminase has been described (e.g. Montiel-Gonzalez et al. PNAS 2013, 110(45):18285-18290; Vogel et al. 2014. Angewandte Chemie Int Ed 53:267-271; Woolf et al. 1995. PNAS 92:8298-8302). Montiel-Gonzalez et al. (2013) described the editing of a target RNA using a genetically engineered fusion protein, comprising an adenosine deaminase domain of the hADAR2 protein fused to a bacteriophage lambda N protein, which recognises the boxB RNA hairpin sequence. The natural dsRNA binding domains of hADAR2 had been removed to eliminate the substrate recognition properties of the natural ADAR and replace it by the boxB recognition domain of lambda N-protein. The authors created an antisense oligonucleotide comprising a ‘guide RNA’ (gRNA) part that is complementary to the target sequence for editing, fused to a boxB portion for sequence specific recognition by the N-domain-deaminase fusion protein. By doing so, it was elegantly shown that the guide RNA oligonucleotide faithfully directed the adenosine deaminase fusion protein to the target site, resulting in guide RNA-directed site-specific A to I editing of the target RNA. These guide RNAs are longer than 50 nucleotides, which is generally too long for therapeutic applications, because of difficulties in manufacturing and limited cell entry. A disadvantage of this method in a therapeutic setting is also the need for a fusion protein consisting of the boxB recognition domain of bacteriophage lambda N-protein, genetically fused to the adenosine deaminase domain of a truncated natural ADAR protein. It requires target cells to be either transduced with the fusion protein, which is a major hurdle, or that target cells are transfected with a nucleic acid construct encoding the engineered adenosine deaminase fusion protein for expression. The latter requirement constitutes no minor obstacle when editing is to be achieved in a multicellular organism, such as in therapy against human disease to correct a genetic disorder.

Vogel et al. (2014) disclosed editing of RNA coding for eCFP and Factor V Leiden, using a benzylguanine substituted guide RNA and a genetically engineered fusion protein, comprising the adenosine deaminase domains of ADAR1 or ADAR2 (lacking the dsRNA binding domains) genetically fused to a SNAP-tag domain (an engineered O6-alkylguanine-DNA-alkyl transferase). Although the genetically engineered artificial deaminase fusion protein could be targeted to a desired editing site in the target RNAs in HeLa cells in culture, through its SNAP-tag domain which is covalently linked to a guide RNA through a 5′-terminal O6-benzylguanine modification, this system suffers from similar drawbacks as the genetically engineered ADARs described by Montiel-Gonzalez et al. (2013), in that it is not clear how to apply the system without having to genetically modify the ADAR first and subsequently transfect or transduct the cells harboring the target RNA, to provide the cells with this genetically engineered protein. Clearly, this system is not readily adaptable for use in humans, e.g. in a therapeutic setting.

Woolf et al. (1995) disclosed a simpler approach, using relatively long single stranded antisense RNA oligonucleotides (25-52 nucleotides in length) wherein the longer oligonucleotides (34-mer and 52-mer) could promote editing of the target RNA by endogenous ADAR because of the double stranded nature of the target RNA and the oligonucleotide hybridizing thereto. The oligonucleotides of Woolf et al. (1995) that were 100% complementary to the target RNA sequences only appeared to function in cell extracts or in amphibian (Xenopus) oocytes by microinjection, and suffered from severe lack of specificity: nearly all adenosines in the target RNA strand that was complementary to the antisense oligonucleotide were edited. An oligonucleotide, 34 nucleotides in length, wherein each nucleotide carried a 2′-O-methyl modification, was tested and shown to be inactive in Woolf et al. (1995). In order to provide stability against nucleases, a 34-mer RNA, modified with 2′-O-methyl-modified phosphorothioate nucleotides at the 5′- and 3′-terminal 5 nucleotides, was also tested. It was shown that the central unmodified region of this oligonucleotide could promote editing of the target RNA by endogenous ADAR, with the terminal modifications providing protection against exonuclease degradation. Woolf et al. (1995) does not achieve deamination of a specific target adenosine in the target RNA sequence. As mentioned, nearly all adenosines opposite an unmodified nucleotide in the antisense oligonucleotide were edited (therefore nearly all adenosines opposite nucleotides in the central unmodified region, if the 5′- and 3′-terminal 5 nucleotides of the antisense oligonucleotide were modified, or nearly all adenosines in the target RNA strand if no nucleotides were modified).

It is known that ADAR may act on any dsRNA. Through a process sometimes referred to as ‘promiscuous editing’, the enzyme will edit multiple A's in the dsRNA. Hence, there is a need for methods and means that circumvent such promiscuous editing and that only target specified adenosines in a target RNA sequence for therapeutic applicability. Vogel et al. (2014) showed that such off-target editing can be suppressed by using 2′-O-methyl-modified nucleotides in the oligonucleotide at positions opposite to the adenosines that should not be edited, and use a non-modified nucleotide directly opposite to the specifically targeted adenosine on the target RNA. However, the specific editing effect at the target nucleotide has not been shown to take place in that article without the use of recombinant ADAR enzymes that have covalent bonds with the antisense oligonucleotide.

WO 2016/097212 discloses antisense oligonucleotides (AONs) for the targeted editing of RNA, wherein the AONs are characterized by a sequence that is complementary to a target RNA sequence (therein referred to as the ‘targeting portion’) and by the presence of a stem-loop structure (therein referred to as the ‘recruitment portion’), which is preferably non-complementary to the target RNA. Such oligonucleotides are referred to as ‘self-looping AONs’. The recruitment portion acts in recruiting a natural ADAR enzyme present in the cell to the dsRNA formed by hybridization of the target sequence with the targeting portion. Due to the recruitment portion there is no need for conjugated entities or presence of modified recombinant ADAR enzymes. WO 2016/097212 describes the recruitment portion as being a stem-loop structure mimicking either a natural substrate (e.g. the GluB receptor) or a Z-DNA structure known to be recognized by the dsRNA binding regions of ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion as described in WO 2016/097212 is an intramolecular stem-loop structure, formed within the AON itself, and able to attract ADAR.

WO 2017/220751 and WO 2018/041973 describe AONs that do not comprise a recruitment portion but that are (almost fully) complementary to the targeted area, except for one or more mismatches, or so-called ‘wobbles’ or bulges. The sole mismatch may be the nucleotide opposite the target adenosine, but in other embodiments AONs are described that have multiple bulges and/or wobbles when attached to the target sequence area. It appeared that it was possible to achieve in vitro, ex vivo and in vivo RNA editing with AONs lacking a recruitment portion and with endogenous ADAR enzymes when the sequence of the AON was carefully selected such that it could attract ADAR. The nucleotide in the AON directly opposite the target adenosines was described as not carrying a 2′-O-methyl modification. It could also be a DNA nucleotide, wherein the remainder of the AON was carrying 2′-O-alkyl modifications at the sugar entity (such as 2′-O-methyl), or the nucleotides within the so-called ‘Central Triplet’ or directly surrounding the Central Triplet contained particular chemical modifications (or were DNA) that further improved the RNA editing efficiency and/or increased the resistance against nucleases. Such effects could even be further improved when using sense oligonucleotides (SONs) that ‘protect’ the AONs against breakdown (described in WO2018/134301).

It is further noted that yet another editing technique exists which uses oligonucleotides, known as the CRISPR/Cas9 system. However, this editing complex in its natural state acts on DNA. It also suffers from the same drawback as the engineered ADAR systems described above, because it requires co-delivery to the target cell of the CRISPR/Cas9 enzyme, or an expression construct encoding the same, together with the guide oligonucleotide. Several investigators are experimenting with base editing of DNA or RNA sequences, for example by employing fusion proteins comprising Cas9 and enzymes with deaminase activity that are guided to the DNA or RNA target site by guide RNAs that are designed in accordance with the CRISPR/Cas9 target finding rules.

Despite the achievements outlined above, there remains a need for new compounds that can utilise (endogenous) cellular pathways and enzymes that have deaminase activity, such as naturally expressed ADAR enzymes to more specifically and more efficiently edit endogenous nucleic acids in mammalian cells, even in whole organisms, to alleviate disease.

### SUMMARY OF THE INVENTION

According to a first aspect, the invention provides an editing oligonucleotide (EON) capable of forming a double stranded complex with a target nucleic acid molecule in a cell, and capable of recruiting an enzyme with nucleotide deaminase activity, wherein the target nucleic acid molecule comprises a target nucleotide for deamination by the enzyme with nucleotide deamination activity, wherein the EON comprises a nucleotide, referred to as nucleotide position 0, which is opposite the target nucleotide and which forms a mismatch with the target nucleotide, and wherein the internucleotide linkage numbering is such that linkage number 0 is the linkage 5′ from nucleotide position 0, and wherein the nucleotide positions and the linkage positions in the EON are both positively (+) and negatively (−) incremented towards the 5′ and 3′ ends, respectively, characterized in that (i) the EON comprises at least one phosphonoacetate internucleotide linkage and at least one internucleotide linkage that is not a phosphonoacetate internucleotide linkage, and/or (ii) the EON comprises at least one nucleotide comprising an unlocked nucleic acid (UNA) ribose modification and at least one nucleotide not comprising a UNA ribose modification.

For the oligonucleotide to qualify as an editing oligonucleotide, the modifications must not be in positions that prevent editing.

In one embodiment the at least one phosphonoacetate internucleotide linkage is at linkage position +19, +18, +17, +16, +15, +14, +10, +9, +5, +4, +3, +2, +1, 0, −6, −7, −8, −9, −10, −11 and/or −12. Preferably, there is not a phosphonoacetate internucleotide linkage at linkage position +13, +12, +11, +8, +7, +6, −1, −2, −3, −4 and/or −5. The internucleotide linkages that are not phosphonoacetate internucleotide linkages are preferably internucleotide linkages independently selected from phosphorothioate, phosphodithioate, 3′-methylenephosphonate, 5′-methylenephosphonate, and/or 3′-phosphoroamidate.

The EON can also comprise a UNA ribose modification at position +19, +18, +17, +16, +15, +11, +10, +9, +8, +7, +6, +5, +4, +3, +1, −1, −2, −4, −5, −6, −7, −8, −9, −10, −11 and/or −12. Preferably, the EON does not comprise a UNA ribose modification at position +14, +13, +12, +2, 0, and/or −3, more preferably the EON does not comprise a UNA ribose modification at position 0. It is also preferred that the EON does not comprise UNA ribose modifications at consecutive positions.

The EON can comprise at least one phosphonoacetate internucleotide linkage, without having any nucleotides comprising a UNA ribose modification. Equally, the EON can comprise a at least one nucleotide comprising a UNA ribose modification, without having any phosphonoacetate internucleotide linkages. Alternatively, the EON can comprise both (i) at least one phosphonoacetate internucleotide linkage, and (ii) at least one nucleotide comprising a UNA ribose modification.

The EON can comprise one or more nucleotides comprising a 2′-O-methoxyethyl (2′-MOE) ribose modification, wherein the EON comprises one or more nucleotides not comprising a 2′-MOE ribose modification. The EON can comprise 2′-O-methyl (2′-OMe) ribose modifications or deoxynucleotides at positions that do not comprise a 2′-MOE ribose modification.

The enzyme with nucleotide deaminase activity preferably comprises a deaminase domain with adenosine deamination activity, and is preferably ADAR1 or ADAR2, more preferably ADAR2. The enzyme with nucleotide deaminase activity is preferably a naturally expressed eukaryotic adenosine deamination enzyme.

The EON is preferably at least 10, 11, 12, 13, 14, 15, 16, 17, 18, 19, 20, 21, 22, 23, 24, 25, 26, 27, 28, 29, 30, 31, or 32 nucleotides in length, and preferably also shorter than 100 nucleotides, preferably shorter than 60 nucleotides.

The target nucleotide is preferably an adenosine that is deaminated to an inosine. The adenosine is preferably located in a UGA or UAG stop codon. It is preferred that the UGA or UAG codon is edited to a UGG codon.

The target nucleic acid molecule is preferably RNA, and the EON is preferably complementary RNA (comprising 2′-OH or 2′-modified ribofuranosyl moieties). The complementary RNA may also comprise one or more 2′-H modifications (i.e. DNA). In a preferred embodiment, two out of three nucleotides in the central triplet are DNA.

The nucleotide opposite the target nucleotide forms a mismatch with the target nucleotide. In one embodiment, the nucleotide opposite the target is an abasic “nucleotide” (i.e. the ribose does not have a base).

According to a second aspect, the invention provides a pharmaceutical composition comprising the EON according to the first aspect of the invention.

According to a third aspect, the invention provides the EON according to the first aspect, or the pharmaceutical composition according to the second aspect, for use in the treatment or prevention of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, ß-thalassemia, CADASIL syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt Disease, Tay-Sachs Disease, Usher syndrome, X-linked immunodeficiency, Sturge-Weber Syndrome, and cancer.

According to a fourth aspect, the invention provides the use of the EON according to the first aspect, or the pharmaceutical composition according to the second aspect, in the manufacture of a medicament for the treatment or prevention of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, ß-thalassemia, CADASIL syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt Disease, Tay-Sachs Disease, Usher syndrome, X-linked immunodeficiency, Sturge-Weber Syndrome, and cancer.

According to a fifth aspect, the invention provides a method for the deamination of at least one target nucleotide present in a target nucleic acid molecule in a cell, the method comprising the steps of: (i) providing the cell with an EON according to the first aspect, or a composition according to the second aspect of the invention; (ii) allowing uptake by the cell of the oligonucleotide; (iii) allowing annealing of the oligonucleotide to the target nucleic acid molecule; (iv) allowing a mammalian enzyme with nucleotide deaminase activity to deaminate the target nucleotide in the target nucleic acid molecule; and (v) optionally identifying the presence of the deaminated nucleotide in the target nucleic acid molecule.

The invention also provides a method of treating or preventing a genetic disorder in a patient in need thereof, the method comprising administering a therapeutically effective amount of the EON according to the first aspect of the invention, or administering a therapeutically effective amount of the composition according to the second aspect of the invention, to the patient. The patient can be a human or an animal. Preferably the patient is a mammal. More preferably the patient is a human patient. The genetic disorder is preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, ß-thalassemia, CADASIL syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt Disease, Tay-Sachs Disease, Usher syndrome, X-linked immunodeficiency, Sturge-Weber Syndrome, and cancer.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows a schematic representation of a section of RNA, wherein the internucleotide linkage is a phosphonoacetate linkage;

FIG. 2 shows a schematic representation of a section of RNA, wherein the ribose comprises an unlocked nucleic acid (UNA) ribose modification;

FIG. 3 shows the primary sequence of an EON encompassing the binding region of the ADAR2 deaminase domain and one of the dsRNA binding domains. The sequence in this particular instance (5′ to 3′; agccuuugag accucugccc agaguuguuc uc; SEQ ID NO: 1) is complementary to the mouse Idua RNA target. Upper numbers are used to characterize linkage positions whereas numbers below correspond to nucleotide positions. The “x” and “—” notification is the mapping of the tolerability for a position-based phosphonoacetate linkage insertion, within a bound editing oligonucleotide, generated through structure-based computational modelling. The notification “x” indicates positions where chemical modification to a phosphonoacetate internucleotide linkage should preferably be avoided because it would significantly disrupt the protein-RNA interaction. The notification “—” indicates positions where chemical modification to a phosphonoacetate internucleotide linkage is not recommended because it would moderately disrupt the protein-RNA interaction. A blank space indicates that the chemical modification to a phosphonoacetate internucleotide linkage would not interfere with the protein-RNA interaction.

FIG. 4 shows the same primary sequence as FIG. 3. The middle C of the central triplet is arbitrarily referenced as 0, positively (+) and negatively (−) incremented towards the 5′ and 3′ ends of the EON, respectively. The box shading is the mapping of the tolerability for a position-based unlocked nucleic acid insertion, within a bound editing oligonucleotide, generated through structure-based computational modelling. Black boxes indicate the positions where a UNA modification should preferably be avoided because it would significantly disrupt the protein-RNA interaction. Grey boxes indicate the positions where UNA modifications are not recommended, because they would moderately disrupt the protein-RNA interaction. White boxes represent the locations where the insertion of UNA modifications does not interfere with the EON-ADAR2 deaminase domain interaction.

FIG. 5 is a representative atomic-scale model generated with the structure-based computational approach. The protein cartoon representation 2 shows the human ADAR2 protein deaminase domain 4 and its double-stranded RNA binding domain 6. The bound double-stranded RNA 8 is constituted by the IDUA gene, shown with light grey sticks, annealed to the editing oligonucleotide 10 displayed with dark grey sticks. The 3 nucleotides composing the central triplet 12 are highlighted with a dark shading of the ribose subunits. Thin continuous lines connecting phosphate atoms are shown to better visualise the helical structure of the RNA.

FIG. 6 is a representative atomic-scale model showing potential recurrent steric hindrances between the human ADAR2 protein 2 sidechains and a phosphonoacetate group 14 inserted between positions 13 and 14 of the bound editing oligonucleotide 10 (position 13 according to linkages numbering). The chemically modified editing oligonucleotide 10 is displayed as sticks and, for clarity, the annealed targeted RNA is not shown. The backbone of the modelled ADAR2 deaminase domain 4 is in cartoon representation, with the lysine 97 side chain 16 from the ADAR2 dsRBD shown with sticks. A thin continuous line connecting phosphate atoms is shown to better visualise the helical structure of the RNA.

FIG. 7 is a representative atomic-scale model showing the lack of hydrogen bond formation between the human ADAR2 protein 2 side-chain and the 2′-O of an unlocked nucleotide 18 inserted at position 0 of the bound editing oligonucleotide 10. The dashed lines between the arginine 510 side-chain 20 amide groups of ADAR2 deaminase domain 4 and the 2′-O of the modified nucleotide 18 characterize distances of 4 angstroms and above. The chemically modified editing oligonucleotide 10 is displayed as sticks and, for clarity, the annealed target RNA is not shown. Nucleotides of the central triplet 12 opposite the edited adenine are shown with dark shading. The backbone of the modelled ADAR2 deaminase domain 4 is in cartoon representation, with the sidechain of arginine 510 displayed with sticks.

FIG. 8 shows the sequence and chemical modifications of the EONs tested. For each EON, the nucleotide sequence is first shown 5′ to 3′, with the chemical modifications indicated as follows: RNA is indicated by capital letters, 2′-O-methyl (2′-OMe) modified nucleotides are shown in lowercase letters, UNA is shown in bolded italic capital letters, and phosphorothioate linkages are indicated by asterisks. Below the EON sequence, another schematic shows the complementarity of the EON to the GFP-stop target sequence, where applicable. Here, a portion of the sequence of the target RNA is shown 5′ to 3′ (upper strand in each panel), with the target adenosine (A) underlined. The sequence of the oligonucleotides in the lower strand is shown from 3′ to 5′. The mismatches (or as sometimes herein referred to as ‘bulges’) between the EON and the RNA are indicated by showing them further below or above the main sequence, respectively. EONs labeled ADAR59-2 and 59-20 target the GFP stop codon, while the EON 65-1 is a negative control EON targeting an unrelated sequence (mouse Idua mRNA with the W392X mutation).

FIG. 9 shows the efficiency of editing, assayed by sequence analysis. The chromatograms show the nucleotide frequency at the target site (highlighted above the center A) and neighboring nucleotides. The nucleotide identity of the peaks is indicated by the same color as in the sequence above the chromatograms, wherein G's are represented in black. The indicated EONs were used at 100 nM concentrations. There is a clear increase in G signal above the central A (shown as a ‘shoulder’ in the neighboring G signal, whereas no shoulder is observed in the control EON ADAR65-1), which shows that both EONs targeting the GFP stop codon RNA induce editing (ADAR59-2 and ADAR59-20).

### DETAILED DESCRIPTION

There is a constant need for improving the pharmacokinetic properties of editing oligonucleotides (EONs) without negatively affecting editing efficiency of the target adenosine in the target nucleic acid. Many chemical modifications exist in the generation of antisense oligonucleotides, whose properties are incompatible with the desire of designing effective EONs. In the search for better pharmacokinetic properties, previously it was found that a 2′-O-methoxyethyl (2′-methoxyethoxy, or 2′-MOE) modification of the ribose of some, but not all, nucleotides surprisingly appeared compatible with efficient ADAR engagement and editing (GB 1802392.9 unpublished). In a similar fashion, it was found that a phosphorothioate linkage at some, but not all, internucleotide linkages surprisingly appeared compatible with efficient ADAR engagement and editing (GB 1808146.3 unpublished). In the search for better pharmacokinetic properties, the inventors of the present invention found that phosphonoacetate linkage modifications and/or unlocked nucleic acid (UNA) ribose modifications of some, but not all, positions in the EON—surprisingly—are compatible with efficient engagement of an enzyme with nucleotide deamination activity and with subsequent deamination. Examples of enhanced pharmacokinetic properties are cellular uptake and intracellular trafficking, stability and so on. Whereas the properties of phosphonoacetate and UNA modifications are known as such, the compatibility thereof with engagement with enzymes with nucleotide deamination activity and with the deamination reaction was not known. The inventors of the present invention have unraveled the positions inside the oligonucleotide where phosphonoacetate linkages and/or UNA ribose modifications are compatible with enzymes with nucleotide deamination activity, the positions where they are not compatible and the positions where they are only compatible to a lesser extent. In an EON, the modifications are not in positions that would prevent editing. These findings can, in principle, be used with any form of base editing employing synthetic oligonucleotides involving, for example, ADAR or ADAR deaminase domains, be they natural or recombinant, truncated or full length, fused to other proteins or not (e.g. Stafforst and Schneider, 2012, Angew Chem Int 51:11166-11169; Schneider et al. 2014, Nucleic Acids Res 42:e87; Montiel-Gonzalez et al. 2016, Nucleic Acids Res 44:e157). The skilled person is aware of a variety of enzymes that have nucleotide deaminase activity, such as ADAR1, ADAR2, APOBEC, Cas13 and the like. The invention, albeit modelled with ADAR2, is not restricted thereto, as the teaching of the current disclosure means that the skilled person can further refine any given EON towards any specific enzyme with nucleotide deaminase activity. Also, therefore, where exemplified herein is an IDUA targeting EON sequence, the teaching of the positions is applicable to any given EON sequence, targeting any other kind of target sequence.

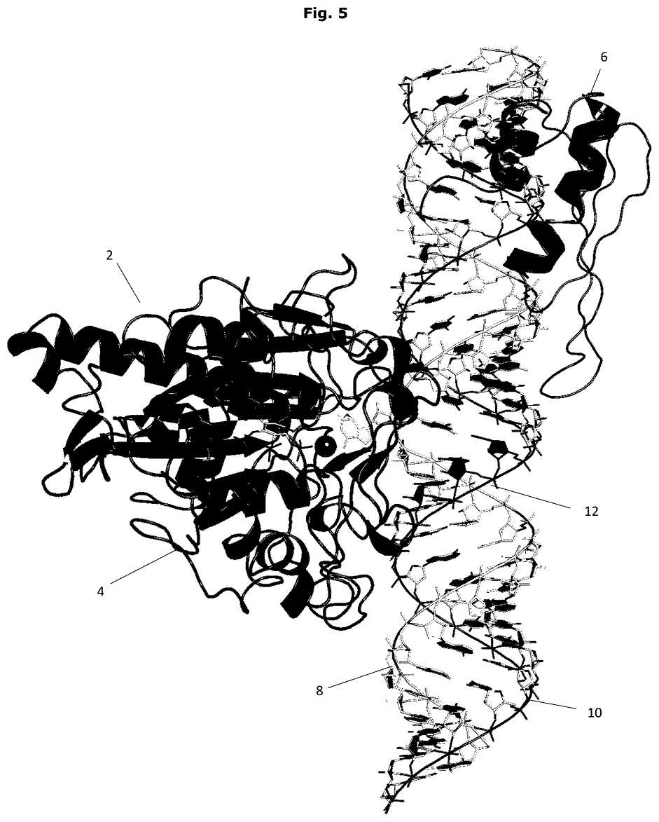

The inventors interrogated EONs with regard to the tolerability of phosphonoacetate internucleosidic linkages and unlocked nucleic acid (UNA) ribose modifications. More in particular, the inventors asked where in the EON phosphonoacetate linkages and UNA ribose modifications are tolerated by the enzyme having deamination activity when interacting with the EON in a helical complex with the target nucleic acid. In the following sections, a more detailed description of the findings and conclusions will be presented based on the interaction of chemically modified EONs designed to bind the target nucleic acid. A specific example involves binding RNA at the target site surrounding a target adenosine, recruiting an adenosine deaminase acting on RNA (ADAR) for deamination of the target adenosine, and converting it into an inosine. FIG. 5 is a representative atomic-scale model generated with the structure-based computational approach. The protein cartoon representation shows the human ADAR2 protein 2 deaminase domain 4 and its double-stranded RNA binding domain 6. The bound double-stranded RNA is constituted by the IDUA gene 8, shown with light grey sticks, annealed to the editing oligonucleotide 10 displayed with dark grey sticks. The 3 nucleotides composing the central triplet 12 are highlighted with a dark shading of the ribose subunits. Thin continuous lines connecting phosphate atoms are shown to better visualise the helical structure of the RNA. It should, however, be clear that the invention is not limited to EONs or methods designed to recruit ADAR to convert target adenosine into inosines. It should be understood, that the invention encompasses any EON that can bind to a target nucleic acid, recruit any protein (naturally expressed proteins as well as foreign proteins, including fusion proteins of different or the same origin) with nucleotide (including adenosine and cytidine) deamination activity, as long as at least one internucleosidic linkage comprises a phosphonoacetate linkage and/or at least one nucleotide comprises a UNA ribose modification.

Editing Oligonucleotides (EONs)

The present invention relates to an EON comprising nucleotides that are linked by internucleosidic linkages, wherein said EON—when forming a double stranded nucleic acid structure by binding to a complementary nucleic acid sequence—is capable of recruiting an enzyme with nucleotide deaminase activity on a target nucleotide in said complementary nucleic acid sequence, characterized in that said EON has been optimized for intramolecular interactions (in particular, hydrogen bonding interactions and steric clashes) between said EON and said enzyme having nucleotide deaminase activity.

The skilled person knows that an oligonucleotide, such as an RNA oligonucleotide, generally consists of repeating monomers. Such a monomer is most often a nucleotide or a nucleotide analogue. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide. A modification in the pentose sugar is therefore often referred to as a “scaffold modification”. For severe modifications, the original pentose sugar might be replaced in its entirety by another moiety that similarly connects the base and the phosphate. It is therefore understood that while a pentose sugar is often a scaffold, a scaffold is not necessarily a pentose sugar.

A base, sometimes called a nucleobase, is generally adenine, cytosine, guanine, thymine or uracil, or a derivative thereof. Cytosine, thymine and uracil are pyrimidine bases, and are generally linked to the scaffold through their 1-nitrogen. Adenine and guanine are purine bases and are generally linked to the scaffold through their 9-nitrogen.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked monomers of an oligonucleotide is often called the “backbone” of the oligonucleotide. Because phosphodiester bonds connect neighboring monomers together, they are often referred to as “backbone linkages”. It is understood that when a phosphate group is modified so that it is instead an analogous moiety such as a phosphorothioate, such a moiety is still referred to as the backbone linkage of the monomer. This is referred to as a “backbone linkage modification”. In general terms, the backbone of an oligonucleotide comprises alternating scaffolds and backbone linkages.

Phosphonoacetate Linkages

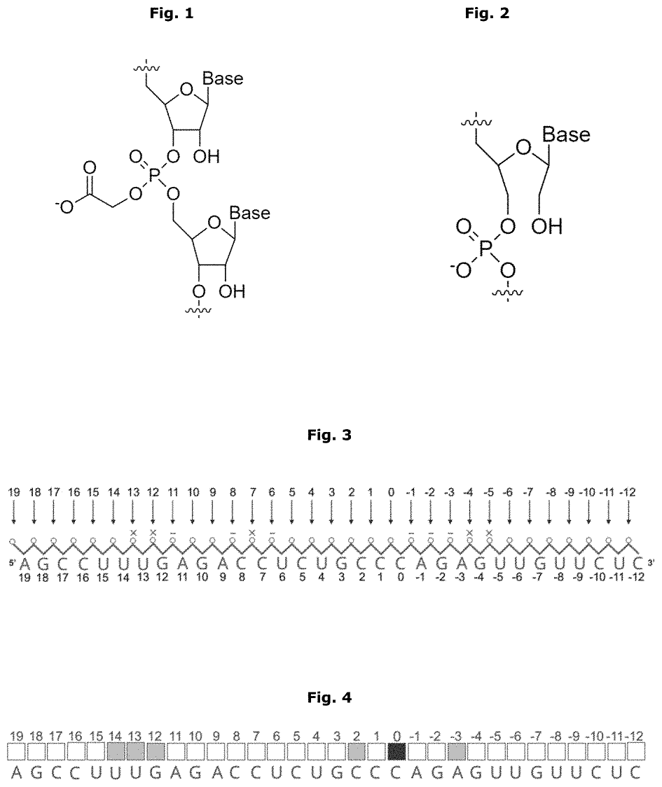

One aspect of the invention relates to an EON that has been optimized for binding to an enzyme with nucleotide deamination activity by placing at least one phosphonoacetate internucleosidic linkage in a position which is not incompatible with editing activity of the enzyme having nucleotide deaminase activity. These binding interactions include optimisation of hydrogen bonding interactions and avoidance of steric clashes. In a preferred embodiment, the EON has been optimized for hydrogen bonding interactions by inserting a phosphonoacetate internucleosidic linkage in one or more positions that favour a stable hydrogen bonding interaction with the enzyme having nucleotide deaminase activity. Phosphonoacetate linkages are well referenced and highly relevant RNA modifications for therapeutically optimized oligonucleotides. The chemical structure of the phosphonoacetate linkage is shown in FIG. 1. An important role of this modification is to protect the polymer from nuclease-mediated degradation. Phosphonoacetate modifications can also be responsible for improved uptake of the EONs into target cells.

Binding of an EON within an enzyme with nucleotide deamination activity is most probably mediated by non-specific contacts. In biological molecules the substitution of the oxygen-phosphate with an acetate group may interfere with intermolecular hydrogen bond networks established in natural protein/RNA complexes. In addition to the fact that the relative angle between two polar groups defines the bond strength, the inventors of the present invention considered in their approach that electronegativity is a major parameter for hydrogen bond formation. Thus, for this structure-based oligonucleotide design, it was assumed that potential hydrogen bond contacts between the EON oxygen-phosphate backbone and the ADAR2 deaminase domain side-chains should be preserved. The inventors of the present invention propose that these connections better support the interaction between the two molecular partners. Phosphonoacetate linkages show potential to fine-tune the interaction between an enzyme and its ligand.

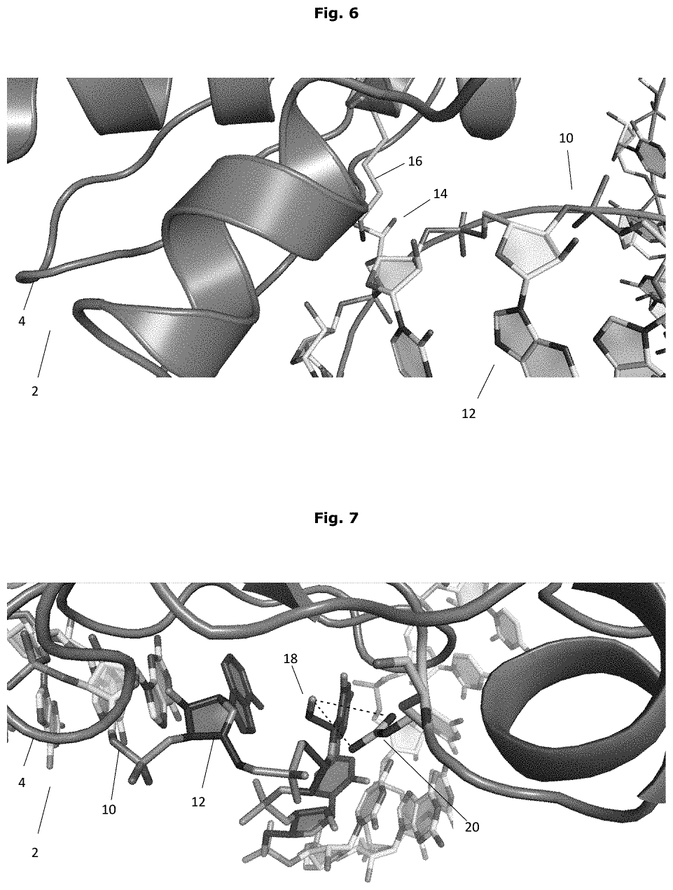

The method allowed the inventors to highlight a pattern of phosphonoacetate linkages compatible with an optimized intermolecular hydrogen bond network between the deaminase domain of the enzyme with ADAR activity (preferably ADAR2) and the EON. To alleviate ambiguities regarding the position of the phosphonoacetate linkage relative to its nucleoside, the selected nomenclature within an extended region of the EON are provided in detail in FIG. 3. Notably, in FIG. 3 the nucleotide opposite the target adenosine in the target sequence is given as the “0” nucleotide position, while the “0” position for the linkage numbering is shifted halfway between nucleotides towards the 5′ end. It is to be understood that the ultimate ‘linkage’ +19 is not linked in this particular example, but may be linked to a next nucleotide in any given EON if it is longer at the 5′ terminus. The same is true for the 3′ end, where additional nucleotides may be attached. The “x” and “—” notification is the mapping of the tolerability for a position-based phosphonoacetate linkage insertion, within a bound editing oligonucleotide, generated through structure-based computational modelling. The notification “x” indicates positions where chemical modification to a phosphonoacetate internucleotide linkage should preferably be avoided because it would significantly disrupt the protein-RNA interaction. The notification “—” indicates positions where chemical modification to a phosphonoacetate internucleotide linkage is not recommended because it would moderately disrupt the protein-RNA interaction. A blank space indicates that the chemical modification to a phosphonoacetate internucleotide linkage would not interfere with the protein-RNA interaction. FIG. 6 shows an example of the determination of the tolerance for a phosphonoacetate modification at position 13. Specifically, FIG. 6 is a representative atomic-scale model showing potential recurrent steric hindrances between the human ADAR2 protein 2 sidechains and a phosphonoacetate group 14 inserted between positions 13 and 14 of the bound editing oligonucleotide 10 (position 13 according to linkages numbering). The chemically modified editing oligonucleotide 10 is displayed as sticks and, for clarity, the annealed targeted RNA is not shown. The backbone of the modelled ADAR2 deaminase domain 4 is in cartoon representation, with the lysine 97 side chain 16 from the ADAR2 dsRBD shown with sticks. For modeling both R and S stereoisomers within the phosphodiester linkages were considered for the insertion of the chemical modification. A thin continuous line connecting phosphate atoms is shown to better visualise the helical structure of the RNA.

In a preferred embodiment, the at least one phosphonoacetate internucleotide linkage is at linkage position +19, +18, +17, +16, +15, +14, +10, +9, +5, +4, +3, +2, +1, 0, −6, −7, −8, −9, −10, −11 and/or −12. In one embodiment, 1, 2, 3, 4, 5, 6, 7, 8, 9, 10 or more of these linkage positions comprise a phosphonoacetate internucleotide linkage. In a preferred embodiment, two or more, preferably five or more, more preferably 10 or more, of these linkage positions comprise a phosphonoacetate internucleotide linkage.

FIG. 3 shows the positions of the linkages and the linkage numbering for (part of) an EON of 25 nucleotides, with linkage numbering 0 is the linkage 5′ of the nucleotide referred to as 0 in the nucleotide numbering. Preferably, using this numbering for linkages, the linkages with number +13, +12, +11, +8, +7, +6, −1, −2, −3, −4 and/or −5 do not have a phosphonoacetate linkage. In one embodiment, the phosphonoacetate linkage is not present in 1, 2, 3, 4, 5, 6, 7, 8, 9, 10 or more of these positions. The internucleotide linkages that are not phosphonoacetate internucleotide linkages are preferably an unmodified phosphodiester or another linkage compatible with editing, such as phosphorothioate, phosphodithioate, 3′-methylenephosphonate, 5′-methylenephosphonate, 3′-phosphoroamidate and the like. In case such linkages are incompatible with editing in one stereoisomer, the editing-compatible stereoisomer may be selected (discussed further in British patent application number GB1808146.3).

In one aspect of the invention, at least one internucleotide linkage, when not carrying a phosphonoacetate modification, may be an unmodified phosphodiester linkage. The at least one unmodified phosphodiester is then preferably at linkage position +13, +12, +11, +8, +7, +6, −1, −2, −3, −4 and/or −5. In one embodiment, the unmodified phosphodiester is present in 1, 2, 3, 4, 5, 6, 7, 8, 9, 10 or more of these positions. In a particularly preferred embodiment, the linkages with number +13, +12, +7, −4 and/or −5 do not have a phosphonoacetate linkage. It is preferred that the phosphonoacetate linkage is absent in at least 2, more preferably at least 3, yet more preferably at least 4, and even more preferably in all 5, of these linkage positions.

Typically, there should not be a phosphonoacetate modification at linkage positions +13, +12, +7, −4 or −5 because a phosphonoacetate modification at these positions can cause significant disruption of binding with the enzyme with nucleotide deamination activity. Usually, it is recommended that there should not be a phosphonoacetate modification at linkage positions +11, +8, +6, −1, −2 or −3 because a phosphonoacetate modification at these positions can cause moderate disruption of binding with the enzyme with nucleotide deamination activity. Where a linkage does not have a phosphonoacetate linkage, it is preferred that this linkage is an unmodified phosphodiester linkage. The phosphonoacetate linker modification may be the only modification in the EON. Alternatively, the phosphonoacetate linker modification may exist in addition to different modifications, such as further linker modifications. In particular, there may additionally be at least one phosphorothioate linker modification. The phosphonoacetate modification may also exist in addition to modifications to the ribose 2′ group. The ribose 2′ groups in the EON can be independently selected from 2′-H (i.e. DNA), 2′-OH (i.e. RNA), 2′-OMe, 2′-MOE, 2′-F, or 2′-4′-linked (i.e. a locked nucleic acid or LNA). The 2′-4′ linkage can be selected from linkers known in the art, such as a methylene linker or constrained ethyl linker. In all cases, the modifications should be compatible with editing such that the oligonucleotide fulfils its role as an editing oligonucleotide.

Unlocked Nucleic Acids

Another aspect of the invention relates to an EON that has been optimized for binding to an enzyme with nucleotide deamination activity by making at least one UNA ribose modification in a position which is not incompatible with editing activity of the enzyme having nucleotide deaminase activity. FIG. 2 shows the chemical structure of a UNA ribose modification. In this modification, there is no carbon-carbon bond between the ribose 2′ and 3′ carbon atoms. UNA ribose modifications therefore increase the local flexibility in oligonucleotides. UNAs can lead to effects such as improved pharmacokinetic properties through improved resistance to degradation. UNAs can also decrease toxicity, and may participate in reducing off-target effects. Whereas the properties of UNA ribose modifications are known as such, the compatibility thereof with engagement a nucleotide deamination enzyme and deamination was not known. The inventors of the present invention have unraveled the positions inside the oligonucleotide where UNA ribose modifications are compatible with such enzymes, where they are not, and where they are only compatible to a lesser extent.

A UNA ribose modification may interfere with intermolecular hydrogen bond networks established in natural protein/RNA complexes. In addition to the fact that the relative angle between two polar groups defines the bond strength, the inventors of the present invention considered in their approach that electronegativity is a major parameter for hydrogen bond formation. The inventors thus identified positions at which a UNA ribose modification would interfere with binding either significantly or moderately. FIG. 4 details the positions thus identified. It is to be understood that the ultimate ‘linkage’ +19 is not linked in this particular example, but may be linked to a next nucleotide in any given EON if it is longer at the 5′ terminus. The box shading is the mapping of the tolerability for a position-based unlocked nucleic acid insertion, within a bound editing oligonucleotide, generated through structure-based computational modelling. Black boxes indicate the positions where a UNA modification should preferably be avoided because it would significantly disrupt the protein-RNA interaction. Grey boxes indicate the positions where UNA modifications are not recommended, because they would moderately disrupt the protein-RNA interaction. White boxes represent the locations where the insertion of UNA ribose modifications does not interfere with the interactions between the EON and the computationally modelled ADAR2 domains. FIG. 7 shows an example of the determination of the tolerance for a UNA ribose modification 18 at position 0 of the EON 10. Specifically, FIG. 7 is a representative atomic-scale model showing the lack of hydrogen bond formation between the human ADAR2 protein 2 side-chain and the 2′-O of an unlocked nucleotide 18 inserted at position 0 of the bound editing oligonucleotide 10. The dashed lines between the arginine 510 side-chain 20 amide groups of ADAR2 deaminase domain 4 and the 2′-O of the modified nucleotide characterize distances of 4 angstroms and above. This distance between a hydrogen donor and an acceptor is not compatible with the formation of a potential hydrogen bond interaction (observed in the experimental structure). The chemically modified editing oligonucleotide 10 is displayed as sticks and, for clarity, the annealed target RNA is not shown. Nucleotides of the central triplet 12 opposite the edited adenine are shown with dark shading. The backbone of the modelled ADAR2 deaminase domain is in cartoon representation, with the sidechain of arginine 510 displayed with sticks.

A UNA ribose modification should preferably be avoided at position 0 as disruption of binding with the enzyme with nucleotide deaminase activity would be significant. It is therefore particularly preferred that position 0 does not have a UNA ribose modification. UNA ribose modification at positions +14, +13, +12, +2 and −3 are not recommended, because modification at any of these positions lead to moderate disruption of binding with the enzyme with nucleotide deaminase activity. It is preferred that at least three of these positions, more preferably at least four of these positions, yet more preferably all five of these positions, do not have a UNA ribose modification.

Positions +19, +18, +17, +16, +15, +11, +10, +9, +8, +7, +6, +5, +4, +3, +1, −1, −2, −4, −5, −6, −7, −8, −9, −10, −11 and −12 were identified as being compatible with UNA ribose modification because these positions do not interfere with binding with the enzyme with nucleotide deaminase activity. In one embodiment, 1, 2, 3, 4, 5, 6, 7, 8, 9, 10, 11, 12, 13, 14, 15, 16, 17, 18, 19, 20, 21, 22, 23, 24, 25 or more of these linkage positions comprise a phosphonoacetate internucleotide linkage. In one embodiment, 5 or more of these positions can have a UNA ribose modification. It is preferred that up to 4 of these positions, more preferably up to 3 of these positions, yet more preferably up to 2 of these positions, have a UNA ribose modification. Preferably, the EON has fewer than 10 UNA ribose modifications, more preferably fewer than 5 UNA ribose modifications, yet more preferably fewer than 3 UNA ribose modifications. In one embodiment, the EON has only a single UNA ribose modification. Where the EON comprises more than one UNA ribose modification, it is preferred that the EON does not have two UNA ribose modifications at consecutive positions. In other words, if a position has a UNA modification, it is preferred that the nucleotide immediately upstream and the nucleotide immediately downstream do not comprise UNA ribose modifications. In a preferred embodiment, the positions that do not comprise UNA ribose modifications comprise a ribose with an intact 2′ to 3′ carbon-carbon bond.

The UNA ribose modification may be the only modification in the EON. Alternatively, the UNA ribose modification may exist in addition to different modifications, such as linker modifications, particularly phosphonoacetate and/or phosphorothioate modifications. The UNA modification may also exist in addition to modifications to the ribose 2′ group, either at positions different to the UNA modifications or at the same positions as the UNA modifications. The ribose 2′ groups in the EON can be independently selected from 2′-H (i.e. DNA), 2′-OH (i.e. RNA), 2′-OMe, 2′-MOE, 2′-F, or 2′-4′-linked (i.e. a locked nucleic acid or LNA). The 2′-4′ linkage can be selected from linkers known in the art, such as a methylene linker or constrained ethyl linker. In all cases, the modifications should be compatible with editing such that the oligonucleotide fulfils its role as an editing oligonucleotide. These different modifications are discussed in more detail below.

Combined Phosphonoacetate and UNA Modifications

The EONs of the present invention can have phosphonoacetate internucleotide linker modifications without having any UNA ribose modifications, and vice versa. The EONs of the present invention can alternatively have both phosphonoacetate internucleotide linker modifications and UNA ribose modifications. Where both modifications are present, the phosphonoacetate internucleotide linker modification may further comprise any of the preferred features described for the phosphonoacetate linker modification and, equally, the UNA ribose modification may further comprise any of the preferred features described for the phosphonoacetate linker modification. In any event, the nucleotide opposite the target nucleotide generally comprises a ribose with a 2′-OH group, or a deoxyribose with a 2′-H group.

Further EON Properties General to Both Phosphonoacetate and UNA Modifications

In a preferred embodiment, the enzyme with nucleoside deaminase activity comprises an ADAR2 deaminase domain or a mutant or derivative thereof or fusion protein therewith. In yet another preferred embodiment the invention relates to an EON, wherein the oligonucleotide has been optimized in 2, 3, 4, 5, 6, 7, 8, 9 or 10 internucleosidic linkages and/or UNA ribose modifications.

In one embodiment, the nucleobases in an EON of the present invention can be adenine, cytosine, guanine, thymine, or uracil. In another embodiment, the nucleobases can be a modified form of adenine, cytosine, guanine, or uracil. In another embodiment, the modified nucleobase can be hypoxanthine (the nucleobase in inosine), pseudouracil, pseudocytosine, 1-methylpseudouracil, orotic acid, agmatidine, lysidine, 2-thiouracil, 2-thiothymine, 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine, 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose). The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy)ribosyl sugar. The term ‘nucleoside’ refers to the nucleobase linked to the (deoxy)ribosyl sugar. The term ‘nucleotide’ refers to the respective nucleobase-(deoxy)ribosyl-phospholinker, as well as any chemical modifications of the ribose moiety or the phospho group. Thus the term would include a nucleotide including a locked ribosyl moiety (comprising a 2′-4′ bridge, comprising a methylene group or any other group, well known in the art), a nucleotide including a linker comprising a phosphodiester, phosphotriester, phosphoro(di)thiolate, methylphosphonates, phosphoramidate linkers, and the like. Sometimes the terms adenosine and adenine, guanosine and guanine, cytosine and cytidine, uracil and uridine, thymine and thymidine, inosine and hypoxanthine, are used interchangeably to refer to the corresponding nucleobase, nucleoside or nucleotide. Sometimes the terms nucleobase, nucleoside and nucleotide are used interchangeably, unless the context clearly requires differently.

In yet another embodiment, the EON comprises one or more nucleotides comprising a 2′-O-methoxyethyl (2′-MOE) ribose modification, wherein the EON comprises one or more nucleotides not comprising a 2′-MOE ribose modification, and wherein the 2′-MOE ribose modifications are at positions that do not prevent the enzyme with nucleotide deaminase activity from deaminating the target nucleotide. And in another preferred embodiment, the EON comprises 2′-O-methyl (2′-OMe) ribose modifications at the positions that do not comprise a 2′-MOE ribose modification, and/or wherein the oligonucleotide comprises deoxynucleotides at positions that do not comprise a 2′-MOE ribose modification. The EON may comprise one or more nucleotides comprising a 2′ position comprising 2′-MOE, 2′-OMe, 2′-OH, 2′-deoxy, 2′-F, or a 2′-4′-linkage (i.e. a locked nucleic acid or LNA). The 2′-4′ linkage can be selected from linkers known in the art, such as a methylene linker or constrained ethyl linker. Different 2′ modifications are discussed in further detail in WO2016/097212, WO2017/220751, WO2018/041973, WO2018/134301, GB1808146.3 (unpublished), and PCT/EP2019/053291. In all cases, the modifications should be compatible with editing such that the oligonucleotide fulfils its role as an editing oligonucleotide. Where a position comprises a UNA ribose modification, that position can have a 2′ position comprising the same modifications discussed above, i.e. 2′-MOE, 2′-OMe, 2′-OH, 2′-deoxy, 2′-F, or a 2′-4′-linkage (i.e. a locked nucleic acid or LNA). Again, in all cases, the modifications should be compatible with editing such that the oligonucleotide fulfils its role as an editing oligonucleotide. In all aspects of the invention, the enzyme with nucleotide deaminase activity is preferably ADAR1 or ADAR2. In a highly preferred embodiment, the EON is an RNA editing oligonucleotide that targets a pre-mRNA or an mRNA, wherein the target nucleotide is an adenosine in the target RNA, wherein the adenosine is deaminated to an inosine, which is being read as a guanosine by the translation machinery. In a further preferred embodiment, the adenosine is located in a UGA or UAG stop codon, which is edited to a UGG codon; or wherein two target nucleotides are the two adenosines in a UAA stop codon, which codon is edited to a UGG codon through the deamination of both target adenosines, wherein two nucleotides in the oligonucleotide mismatch with the target nucleic acid. The invention also relates to a pharmaceutical composition comprising the EON as characterized herein, and a pharmaceutically acceptable carrier.

It was found that at certain positions it was in fact preferred that no phosphonoacetate or UNA ribose modification should be introduced as it would hamper the EON-protein interaction. Notably, as indicated herein, modifications are generally introduced to prevent breakdown (and therethrough increase RNA editing efficiency). The skilled person understands that this introduces a balance between better or weaker interaction between EON and ADAR enzyme on the one hand, and on the other hand increased or decreased stability of the EON in vivo. The invention provides, surprisingly, that it is actually possible that certain positions can carry phosphonoacetate and/or UNA ribose modifications. The skilled person is capable of using methodology in vitro as well as in vivo to further refine which of the positions should or should not carry a modification in order to obtain the most efficient RNA editing outcome. This determination is clearly based on the sequence of the EON itself and the target sequence, possibly the structure of the pre-mRNA, the enzyme with ADAR activity that appears to be used (ADAR 1 or ADAR2), the cell in which the RNA editing should occur, etc.

2′-MOE in More Detail

Preferably, the EON according to the invention comprises nucleotides carrying a 2′-MOE ribose modification and, even more preferably, a 2′-O-methyl (2′-OMe) ribose modification at the positions that do not have a 2′-MOE ribose modification. As outlined herein, the EON comprises a nucleotide directly opposite the target adenosine which is referred to as position 0 of the EON nucleotide sequence. Preferably, the EON comprises one or two deoxynucleotides (DNA) at positions −1 and/or 0, wherein the positions are positively (+) and negatively (−) incremented towards the 5′ and 3′ ends of the EON, respectively. Preferably, the EON of the invention does not comprise a 2′-MOE modification at position +14, +13, +12, +6, +2, +1, 0, −1, −2, −3, −4, and/or −5. In one embodiment, the EON of the invention does not comprise a 2′-MOE modification at positions +14, +13, +12, 0, −1, −2 and/or −3. In other words, there may be a 2′-MOE modification at position +6, +2, +1, −4 and/or −5. Typically, there will only be a single 2′-MOE modification at position +6, +2, +1, −4 and/or −5. In one embodiment, the EON of the invention does not comprise a 2′-MOE modification at position 0, −1, −2 and/or −3. In a particularly preferred embodiment, the EON does not comprise a 2′-MOE modification at position −1 and or 0. The preferred locations of these 2′-MOE, 2′-MeO and 2′ deoxy modifications are discussed in further detail in PCT/EP2019/053291.

In one embodiment, an EON of the present invention comprises a 2′-substituted phosphorothioate monomer, preferably a 2′-substituted phosphorothioate RNA monomer, a 2′-substituted phosphate RNA monomer, or comprises 2′-substituted mixed phosphate/phosphorothioate monomers. It is noted that DNA is considered as an RNA derivative in respect of 2′ substitution. An EON of the present invention comprises at least one 2′-substituted RNA monomer connected through or linked by a phosphorothioate or phosphate backbone linkage, or a mixture thereof. The 2′-substituted RNA preferably is 2′-F, 2′-H (DNA), 2′-O-Methyl or 2′-O-(2-methoxyethyl). The 2′-O-Methyl is often abbreviated to “2′-OMe” and the 2′-O-(2-methoxyethyl) moiety is often abbreviated to “2′-MOE”. More preferably, the 2′-substituted RNA monomer in the EON of the present invention is a 2′-OMe monomer, except for the monomer opposite the target adenosine, as further outlined herein, which should not carry a 2′-OMe substitution. In a preferred embodiment of this aspect is provided an EON according to the invention, wherein the 2′-substituted monomer can be a 2′-substituted RNA monomer, such as a 2′-F monomer, a 2′-NH₂ monomer, a 2′-H monomer (DNA), a 2′-O-substituted monomer, a 2′-OMe monomer or a 2′-MOE monomer or mixtures thereof. Preferably, the monomer opposite the target adenosine is a 2′-H monomer (DNA) but may also be a monomer that allows deamination of the target adenosine, other than a 2′-OMe monomer. Preferably, any other 2′-substituted monomer within the EON is a 2′-substituted RNA monomer, such as a 2′-OMe RNA monomer or a 2′-MOE RNA monomer, which may also appear within the EON in combination.

Throughout the application, a 2′-OMe monomer within an EON of the present invention may be replaced by a 2′-OMe phosphorothioate RNA, a 2′-OMe phosphate RNA or a 2′-OMe phosphate/phosphorothioate RNA. Throughout the application, a 2′-MOE monomer may be replaced by a 2′-MOE phosphorothioate RNA, a 2′-MOE phosphate RNA or a 2′-MOE phosphate/phosphorothioate RNA. Throughout the application, an oligonucleotide consisting of 2′-OMe RNA monomers linked by or connected through phosphorothioate, phosphate or mixed phosphate/phosphorothioate backbone linkages may be replaced by an oligonucleotide consisting of 2′-OMe phosphorothioate RNA, 2′-OMe phosphate RNA or 2′-OMe phosphate/phosphorothioate RNA. Throughout the application, an oligonucleotide consisting of 2′-MOE RNA monomers linked by or connected through phosphorothioate, phosphate or mixed phosphate/phosphorothioate backbone linkages may be replaced by an oligonucleotide consisting of 2′-MOE phosphorothioate RNA, 2′-MOE phosphate RNA or 2′-MOE phosphate/phosphorothioate RNA.

Other Modifications in More Detail

In addition to the specific preferred chemical modifications at certain positions in compounds of the invention, compounds of the invention may comprise or consist of one or more (additional) modifications to the nucleobase, scaffold and/or backbone linkage, which may or may not be present in the same monomer, for instance at the 3′ and/or 5′ position. A scaffold modification indicates the presence of a modified version of the ribosyl moiety as naturally occurring in RNA (i.e. the pentose moiety), such as bicyclic sugars, tetra hydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O-(substituted)alkyl such as 2′-O-methyl, 2′-O-(2-cyanoethyl), 2′-MOE, 2′-O-(2-thiomethyl)ethyl, 2′-O-butyryl, 2′-O-propargyl, 2′-O-allyl, 2′-O-(2-aminopropyl), 2′-O-(2-(dimethylamino)propyl), 2′-O-(2-amino)ethyl, 2′-O-(2-(dimethylamino)ethyl); 2′-deoxy (DNA); 2′-O-(haloalkyl)methyl such as 2′-O-(2-chloroethoxy)methyl (MCEM), 2′-O-(2,2-dichloroethoxy)methyl (DCEM); 2′-O-alkoxycarbonyl such as 2′-O-[2-(methoxycarbonyl)ethyl] (MOCE), 2′-O-[2-N-methylcarbamoyl)ethyl] (MCE), 2′-O-[2-(N,N-dimethylcarbamoyl)ethyl] (DCME); 2′-halo e.g. 2′-F, FANA; 2′-O-[2-(methylamino)-2-oxoethyl] (NMA); a bicyclic or bridged nucleic acid (BNA) scaffold modification such as a conformationally restricted nucleotide (CRN) monomer, a locked nucleic acid (LNA) monomer, a xylo-LNA monomer, an α-LNA monomer, an α-I-LNA monomer, a β-d-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-O,4′-C) constrained ethyl (cEt) BNA monomer, a (2′-O,4′-C) constrained methoxyethyl (cMOE) BNA monomer, a 2′,4′-BNA^{NC}(NH) monomer, a 2′,4′-BNA^{NC}(NMe) monomer, a 2′,4′-BNA^{NC}(NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-I-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above. All of these modifications are known to the person skilled in the art.

Other Linkages in More Detail

A “backbone modification” indicates the presence of a modified version of the ribosyl moiety (“scaffold modification”), as indicated above, and/or the presence of a modified version of the phosphodiester as naturally occurring in RNA (“backbone linkage modification”). In particular, the EON according to the invention can comprise internucleoside linkage modifications other than, or in addition to, phosphonoacetate linker modifications. Examples of internucleoside linkage modifications are phosphorothioate (PS), chirally pure phosphorothioate, Rp phosphorothioate, Sp phosphorothioate, phosphorodithioate (PS2), phosphonoacetate (PACE), thophosphonoacetate, phosphonacetamide (PACA), thiophosphonacetamide, phosphorothioate prodrug, S-alkylated phosphorothioate, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methyl boranophosphonothioate, phosphoryl guanidine (PGO), methylsulfonyl phosphoroamidate, phosphoramidite, phosphonamidate, N3′→P5′ phosphoramidate, N3′→P5′ thiophosphoramidate, phosphorodiamidate, phosphorothiodiamidate, sulfamate, dimethylenesulfoxide, sulfonate, triazole, oxalyl, carbamate, methyleneimino (MMI), and thioacetamido (TANA); and their derivatives.

In one embodiment the internucleotide linkage can be a phosphodiester wherein the OH group of the phosphodiester has been replaced by alkyl, alkoxy, aryl, alkylthio, acyl, —NR1R1, alkenyloxy, alkynyloxy, alkenylthio, alkynylthio, —S—Z+, —Se—Z+, or —BH3-Z+, and wherein R1 is independently hydrogen, alkyl, alkenyl, alkynyl, or aryl, and wherein Z+ is ammonium ion, alkylammonium ion, heteroaromatic iminium ion, or heterocyclic iminium ion, any of which is primary, secondary, tertiary or quaternary, or Z is a monovalent metal ion, and is preferably a phosphothioate linkage. In a preferred embodiment the internucleotide linkage modification other than, or in addition to, phosphonoacetate linker modifications is not at position +8, +7, +6, +5, −4 and/or −5. Where a phosphothioate linkage modification is at position 10, it is preferably in the S configuration. Where a phosphothioate linkage modification is at position +4, −1, −3 and/or −6, it is preferably in the R configuration. The preference for specific phosphorothioate stereochemistry at different positions is described further in British patent application number GB1808146.3 (unpublished).

Pharmaceutical Compositions and Medical Uses

The invention also relates to a pharmaceutical composition comprising the EON according to the first aspect of the invention, and a pharmaceutically acceptable carrier. Suitable pharmaceutically acceptable carriers are well known to the person skilled in the art. The invention also relates to an EON according to the first aspect of the invention, or a composition according to the second aspect of the invention, for use in the treatment or prevention of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, ß-thalassemia, CADASIL syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt Disease, Tay-Sachs Disease, Usher syndrome, X-linked immunodeficiency, Sturge-Weber Syndrome, and cancer. The invention also relates to a use of the EON according to the first aspect of the invention, or a composition according to the second aspect of the invention, in the manufacture of a medicament for the treatment or prevention of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, ß-thalassemia, CADASIL syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt Disease, Tay-Sachs Disease, Usher syndrome, X-linked immunodeficiency, Sturge-Weber Syndrome, and cancer.

In yet another embodiment, the invention relates to a method for the deamination of at least one target adenosine present in a target nucleic acid molecule in a cell, the method comprising the steps of providing the cell with an EON according to the first aspect of the invention, or a composition according to the second aspect of the invention, allowing uptake by the cell of the EON, allowing annealing of the EON to the target RNA molecule, allowing a mammalian enzyme with nucleotide deaminase activity to deaminate the target nucleotide in the target nucleic acid molecule, and optionally identifying the presence of the deaminated nucleotide in the target nucleic acid molecule. Preferably, the presence of the target nucleic acid molecule is detected by either (i) sequencing the target sequence, (ii) assessing the presence of a functional, elongated, full length and/or wild type protein when the target adenosine is located in a UGA or UAG stop codon, which is edited to a UGG codon through the deamination, (iii) assessing the presence of a functional, elongated, full length and/or wild type protein when two target adenosines are located in a UAA stop codon, which is edited to a UGG codon through the deamination of both target adenosines, (iv) assessing whether splicing of the pre-mRNA was altered by the deamination; or (v) using a functional read-out, wherein the target RNA after the deamination encodes a functional, full length, elongated and/or wild type protein. Clearly, when two target nucleotides need to be deaminated, the linkage numbering (as outlined herein for a single target adenosine) should be adjusted accordingly.

EONs and their Uses

The EONs of the present invention preferably do not comprise a recruitment portion as described in WO 2016/097212. The EONs of the present invention preferably do not comprise a portion that is capable of forming an intramolecular stem-loop structure. In one embodiment, the present invention relates to EONs that target premature termination stop codons (PTCs) present in the (pre)mRNA to alter the adenosine present in the stop codon to an inosine (read as a G), which in turn then results in read-through during translation and a full length functional protein. In one particular embodiment, the present invention relates to EONs for use in the treatment of cystic fibrosis (CF), and in an even further preferred embodiment, the present invention relates to EONs for use in the treatment of CF wherein PTCs such as the G542X (UGAG), W1282X (UGAA), R553X (UGAG), R1162X (UGAG), Y122X (UAA, both adenosines), W1089X, W846X, and W401X mutations are modified through RNA editing to amino acid encoding codons, and thereby allowing the translation to full length proteins. The teaching of the present invention, the computational modelling of allowable and not-allowable positions regarding phosphonoacetate linkage modifications and/or UNA ribose modifications, as outlined herein, is applicable for all genetic diseases that may be targeted with EONs and may be treated through RNA editing.

In the EON, the nucleotide opposite the target nucleotide generally comprises a ribose with a 2′-OH group, or a deoxyribose with a 2′-H group. Further, the EON generally does not have 2′-MOE modifications at certain positions relative to the nucleotide opposite the target nucleotide, and further does have 2′-MOE modifications at other positions within the EON, as further defined herein. The EON preferably does not comprise a portion that is capable of forming an intramolecular stem-loop structure that is capable of binding an ADAR enzyme. The EON does preferably not include a 5′-terminal 06-benzylguanine modification. The EON preferably does not include a 5′-terminal amino modification. The EON preferably is not covalently linked to a SNAP-tag domain. In another preferred embodiment the target RNA is human CFTR. In a more preferred embodiment, the stop codon is a premature termination stop codon in the human CFTR (pre)mRNA and even more preferably selected from the group of stop codon mutations in CFTR consisting of: G542X, W1282X, R553X, R1162X, Y122X, W1089X, W846X, and W401X. More preferably, the splice mutation in human CFTR is selected from the group of consisting of: 621+1G>T and 1717-1G>A. In one aspect, the present invention relates to an EON for use in the treatment of Cystic Fibrosis, wherein the EON enables the deamination of an adenosine present in a PTC present in the CFTR (pre)mRNA and wherein the PTC results in early translation termination that eventually causes the disease.

In yet another aspect, the invention relates to an EON capable of forming a double stranded complex with a target RNA in a cell, for use in the deamination of a target adenosine in a disease-related splice mutation present in the target RNA, wherein the nucleotide in the EON that is opposite the target adenosine does not carry a 2′-O-methyl (2′-OMe) modification; the nucleotide directly 5′ and/or 3′ from the nucleotide opposite the target adenosine carry a sugar modification and/or a base modification to render the EON more stable and/or more effective in RNA editing. In another preferred aspect the nucleotide in the EON opposite the target adenosine is not RNA but DNA, and in an even more preferred aspect, the nucleotide opposite the target adenosine as well as the nucleotide 5′ and/or 3′ of the nucleotide opposite the target adenosine are DNA nucleotides, while the remainder (not DNA) of the nucleotides in the EON are preferably 2′-O-alkyl modified ribonucleotides. When two nucleotides are DNA all others may be RNA and may be 2′-OMe or 2′-MOE modified, whereas in particular aspects the third nucleotide in the triplet opposite the target adenosine may be RNA and non-modified, as long as the nucleotide opposite the target adenosine is not 2′-OMe modified. In one particular aspect the invention relates to an EON for the deamination of a target adenosine present in the target RNA by an enzyme present in the cell (likely an ADAR enzyme), wherein the EON is (partly) complementary to a target RNA region comprising the target adenosine, wherein the nucleotide opposite the target adenosine comprises a deoxyribose with a 2′-H group, wherein the nucleotide 5′ and/or 3′ of the nucleotide opposite the target adenosine also comprises a deoxyribose with a 2′-H group, and the remainder of the EON comprises ribonucleosides, preferably all with 2′-OMe or 2′-MOE modifications. In the case of two sequential adenosines (e.g. in the Y122X mutation: UAA) that need to be edited, it is preferred that the nucleotides in the EON that are opposite the two adenosines do both not carry a 2′-O-methyl modification. In another preferred aspect, the EON according to the invention is not a 17-mer or a 20-mer. In yet another aspect the EON according to the invention is longer than 17 nucleotides, or shorter than 14 nucleotides. In a preferred embodiment, the EON according to the invention comprises 2, 3, 4, 5, 6, 7, 8, 9 or 10 mismatches, wobbles and/or bulges with the complementary target RNA region. Preferably, the nucleotide is opposite a target adenosine and is a cytidine, a deoxycytidine, a uridine, a deoxyuridine, or is abasic. When the nucleotide opposite the target adenosine is a cytidine or a deoxycytidine, the EON comprises at least one mismatch with the target RNA molecule. When the nucleotide opposite the target adenosine is a uridine or a deoxyuridine, the EON may be 100% complementary and not have any mismatches, wobbles or bulges in relation to the target RNA. However, in a preferred aspect one or more additional mismatches, wobbles and/or bulges are present between EON and target RNA whether the nucleotide opposite the target adenosine is a cytidine, a deoxycytidine, a uridine, or a deoxyuridine. In another preferred embodiment, the nucleotide directly 5′ and/or 3′ from the nucleotide opposite the target adenosine (together with the nucleotide opposite the target adenosine forming a triplet) comprises a ribose with a 2′-OH group, or a deoxyribose with a 2′-H group, or a mixture of these two (triplet consists then of DNA-DNA-DNA; DNA-DNA-RNA; DNA-RNA-DNA; DNA-RNA-RNA; RNA-DNA-DNA; RNA-DNA-RNA; RNA-RNA-DNA; or RNA-RNA-RNA; wherein the middle nucleoside does not have a 2′-O-methyl modification (when RNA) and either or both surrounding nucleosides also do not have a 2′-O-methyl modification). It is then preferred that all other nucleotides in the EON then do have a 2′-O-alkyl group, preferably a 2′-O-methyl group, or a 2′-O-methoxyethyl (2′-MOE) group, or any modification as disclosed herein. The EONs of the present invention comprise at least one phosphonoacetate linkage modification and or at least one UNA ribose modifications. 2′-OMe and 2′-MOEs should not influence the location of the phosphonoacetate or UNA ribose modifications, only global effect on the EON properties may be observed including hydrophobicity, melting temperature, etc. For this, combinations may have an influence. However, for the binding of the deaminase domain, it should not interfere. All mentioned chemical modifications could be applied, in principle, to other disease models, as they involve the backbone not the primary sequence. Calculations should not be systematically repeated with other disease models except if it is shown that affinities can strongly vary between RNA targets. This would suggest that the local binding is shifted.

In one particular embodiment of the present invention, the EON is longer than 10, 11, 12, 13, 14, 15, 16 or 17 nucleotides. Preferably, the EON is shorter than 100 nucleotides, more preferably shorter than 60 nucleotides, and even more preferably, the EON comprises 18 to 70 nucleotides, 18 to 60 nucleotides, or 18 to 50 nucleotides.

A single nucleotide of the EON can have one, or more than one sugar modification. Within the EON, one or more nucleotide(s) can have such sugar modification(s).

It is typically also an important aspect of the invention that the nucleotide within the EON of the present invention that is opposite to the nucleotide that needs to be edited does not contain a 2′-O-methyl modification (herein often referred to as a 2′-OMe group, or as 2′-O-methylation) and preferably comprises a 2′-OH group, or is a deoxyribose with a 2′-H group. The nucleotide in the EON opposite the target nucleotide for editing may also be abasic, i.e. not having a base coupled to the ribofuranosyl moiety. It is preferred that the nucleotides that are directly 3′ and/or 5′ of this nucleotide (the ‘neighbouring nucleotides’) also lack such a chemical modification, although it is believed that it is tolerated that one of these neighbouring nucleotides may contain a 2′-O-alkyl group (such as a 2′-O-methyl group), but preferably not both. Either one, or both neighbouring nucleotides may be 2′-OH or a compatible substitution (as defined herein).

Preferably the EON of the present invention does not have a portion that allows the EON in itself to fold into an intramolecular hairpin or other type of (stem) loop structure (herein also referred to as “auto-looping” or “self-looping”), and which may potentially act as a structure that sequesters ADAR. In one aspect, the single stranded EON of the present invention mismatches with the target nucleotide, and preferably the target is adenosine, where the opposite nucleoside is then preferably a cytidine. The single-stranded RNA editing oligonucleotides of the present invention may also have one or more mismatches, wobbles or bulges (no opposite nucleoside) with the target sequence, at positions other than at the target position. These wobbles, mismatches and/or bulges of the EON of the present invention with the target sequence do not prevent hybridization of the oligonucleotide to the target RNA sequence, but add to the RNA editing efficiency by the ADAR present in the cell, at the target adenosine position. The person skilled in the art is able to determine whether hybridization under physiological conditions still does take place. In contrast to the prior art, the EON of the present invention can recruit a mammalian ADAR enzyme present in the cell, wherein the ADAR enzyme comprises its natural dsRNA binding domain as found in the wild type enzyme. The EONs according to the present invention can utilise endogenous cellular pathways and naturally available ADAR enzyme, or enzymes with ADAR activity (which may be yet unidentified ADAR-like enzymes) to specifically edit a target adenosine in a target RNA sequence. As disclosed herein, the single-stranded EONs of the invention are capable of deamination of a specific target, such as adenosine, in a target nucleic acid, such as RNA, sequence. Ideally, only one nucleotide is deaminated. Alternatively 1, 2, or 3 further nucleotides are deaminated, but preferably only one. Taking the features of the EONs of the present invention together, there is no need for modified recombinant ADAR expression, there is no need for conjugated entities attached to the EON, or the presence of long recruitment portions that are not complementary to the target RNA sequence. Besides that, the EON of the present invention does allow for the specific deamination of a target nucleotide present in the target nucleic acid molecule by a natural nucleotide deaminase enzyme comprising a natural dsRNA binding domain as found in the wild type enzyme, without the risk of promiscuous editing elsewhere in the RNA/EON complex.

Analysis of natural targets of ADAR enzymes indicated that these generally include mismatches between the two strands that form the RNA helix edited by ADAR1 or ADAR2. It has been suggested that these mismatches enhance the specificity of the editing reaction (Stefl et al. 2006. Structure 14(2):345-355; Tian et al. 2011. Nucleic Acids Res 39(13):5669-5681). Characterization of optimal patterns of paired/mismatched nucleotides between the EONs and the target RNA also appears crucial for development of efficient nucleotide deaminase-based EON therapy. An improved feature of the EONs of the present invention is the use of specific phosphonoacetate linkage modifications and/or UNA ribose modifications at predefined spots to ensure stability as well as proper nucleotide deaminase binding and activity. These changes may include further modifications in the backbone of the EON, in the sugar moiety of the nucleotides as well as in the nucleobases. They may also be variably distributed throughout the sequence of the EON, depending on the target and on secondary structures. Specific chemical modifications may be needed to support interactions of different amino acid residues within the RNA-binding domains of ADAR enzymes, as well as those in the deaminase domain. For example, 2′-O-methyl modifications are tolerated in some parts of the EON, while in other parts they should be avoided so as not to disrupt crucial interactions of the enzyme with the phosphate and/or 2′-OH groups. Part of these design rules are guided by the published structures of ADAR2, while others have to be defined empirically. The modifications should also be selected such that they prevent degradation of the EONs. Previous work has established that certain sequence contexts are more amenable to editing. For example, the target sequence 5′-UAG-3′ (with the target A in the middle) contains the most preferred nearest-neighbor nucleotides for ADAR2, whereas a 5′-CAA-3′ target sequence is disfavored (Schneider et al. 2014. Nucleic Acids Res 42(10):e87). The recent structural analysis of ADAR2 deaminase domain hints at the possibility of enhancing editing by careful selection of the nucleotides that are opposite to the target trinucleotide. For example, the 5′-CAA-3′ target sequence, paired to a 3′-GCU-5′ sequence on the opposing strand (with the A-C mismatch formed in the middle in this triplet), is disfavored because the guanosine base sterically clashes with an amino acid side chain of ADAR2. However, here it is postulated that a smaller nucleobase, such as inosine, could potentially fit better into this position without causing steric clashes, while still retaining the base-pairing potential to the opposing cytidine. Modifications that could enhance activity of suboptimal sequences include the use of backbone modifications that increase the flexibility of the EON or, conversely, force it into a conformation that favors editing.

### Definitions of Terms as Used Herein

As set out earlier in this description, the terms ‘adenine’, ‘guanine’, ‘cytosine’, ‘thymine’, ‘uracil’ and ‘hypoxanthine’ (the nucleobase in inosine) as used herein refer to the nucleobases as such.

The terms ‘adenosine’, ‘guanosine’, ‘cytidine’, ‘thymidine’, ‘uridine’ and ‘inosine’, refer to the nucleobases linked to the (deoxy)ribosyl sugar.

The term ‘nucleoside’ refers to the nucleobase linked to the (deoxy)ribosyl sugar.

The term ‘nucleotide’ refers to the respective nucleobase-(deoxy)ribosyl-phospholinker, as well as any chemical modifications of the ribose moiety or the phospho group. Thus the term would include a nucleotide including a locked ribosyl moiety (comprising a 2′-4′ bridge, comprising a methylene group or any other group, well known in the art), an unlocked nucleic acid, a nucleotide including a linker comprising a phosphodiester, phosphonoacetate, phosphotriester, phosphoro(di)thiolate, methylphosphonates, phosphoramidate linkers, and the like.

Sometimes the terms adenosine and adenine, guanosine and guanine, cytidine and cytosine, uracil and uridine, thymine and thymidine/uridine, inosine and hypo-xanthine, are used interchangeably to refer to the corresponding nucleobase on the one hand, and the nucleoside or nucleotide on the other.

Sometimes the terms nucleobase, nucleoside and nucleotide are used interchangeably, unless the context clearly requires differently. The terms ‘ribonucleoside’ and ‘deoxyribonucleoside’, or ‘ribose’ and ‘deoxyribose’ are as used in the art.

Whenever reference is made to an ‘oligonucleotide’, both oligoribonucleotides and deoxyoligoribonucleotides are meant unless the context dictates otherwise. Whenever reference is made to an ‘oligoribonucleotide’ it may comprise the bases A, G, C, U or I. Whenever reference is made to a ‘deoxyoligoribonucleotide’ it may comprise the bases A, G, C, T or I. In a preferred aspect, the EON of the present invention is an oligoribonucleotide that may comprise chemical modifications, and may include deoxynucleotides (DNA) at certain specified positions. Terms such as oligonucleotide, oligo, ON, oligonucleotide composition, antisense oligonucleotide, AON, and RNA (antisense) oligonucleotide may be used herein interchangeably.

Whenever reference is made to nucleotides in the oligonucleotide construct, such as cytosine, 5-methylcytosine, 5-hydroxymethylcytosine and β-D-Glucosyl-5-hydroxymethylcytosine are included; when reference is made to adenine, N6-Methyladenine and 7-methyladenine are included; when reference is made to uracil, dihydrouracil, 4-thiouracil and 5-hydroxymethyluracil are included; when reference is made to guanine, 1-methylguanine is included.

Whenever reference is made to nucleosides or nucleotides, ribofuranose derivatives, such as 2′-desoxy, 2′-hydroxy, and 2′-O-substituted variants, such as 2′-O-methyl, are included, as well as other modifications, including 2′-4′ bridged variants.

Whenever reference is made to oligonucleotides, linkages between two mononucleotides may be phosphodiester linkages as well as modifications thereof, including, phosphonoacetate, phosphodiester, phosphotriester, phosphoro(di)thiolate, methylphosphonate, phosphor-amidate linkers, and the like.

The term ‘comprising’ encompasses ‘including’ as well as ‘consisting’, e.g. a composition ‘comprising X’ may consist exclusively of X or may include something additional, e.g. X+Y.

The term ‘about’ in relation to a numerical value x is optional and means, e.g. x±10%.

The word ‘substantially’ does not exclude ‘completely’, e.g. a composition which is ‘substantially free from Y’ may be completely free from Y. Where relevant, the word ‘substantially’ may be omitted from the definition of the invention.

The term “complementary” as used herein refers to the fact that the AON (or EON as it is often referred to herein) hybridizes under physiological conditions to the target sequence. The term does not mean that each and every nucleotide in the AON has a perfect pairing with its opposite nucleotide in the target sequence. In other words, while an AON may be complementary to a target sequence, there may be mismatches, wobbles and/or bulges between AON and the target sequence, while under physiological conditions that AON still hybridizes to the target sequence such that the cellular RNA editing enzymes can edit the target adenosine. The term “substantially complementary” therefore also means that in spite of the presence of the mismatches, wobbles, and/or bulges, the AON has enough matching nucleotides between AON and target sequence that under physiological conditions the AON hybridizes to the target RNA. As shown herein, an AON may be complementary, but may also comprise one or more mismatches, wobbles and/or bulges with the target sequence, as long as under physiological conditions the AON is able to hybridize to its target.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand, but is downstream of the stop codon in the antisense strand.

References to ‘hybridisation’ typically refer to specific hybridisation and exclude non-specific hybridisation. Specific hybridisation can occur under experimental conditions chosen, using techniques well known in the art, to ensure that the majority of stable interactions between probe and target are where the probe and target have at least 70%, preferably at least 80%, more preferably at least 90% sequence identity.

The term ‘mismatch’ is used herein to refer to opposing nucleotides in a double stranded RNA complex which do not form perfect base pairs according to the Watson-Crick base pairing rules. Mismatched nucleotides are G-A, C-A, U-C, A-A, G-G, C-C, U-U pairs. In some embodiments EONs of the present invention comprise fewer than four mismatches, for example 0, 1 or 2 mismatches. Wobble base pairs are: G-U, I-U, I-A, and I-C base pairs.

The term ‘splice mutation’ relates to a mutation in a gene that encodes for a pre-mRNA, wherein the splicing machinery is dysfunctional in the sense that splicing of introns from exons is disturbed and due to the aberrant splicing the subsequent translation is out of frame resulting in premature termination of the encoded protein. Often such shortened proteins are degraded rapidly and do not have any functional activity, as discussed herein. In a preferred aspect, the splice mutations that are targeted by the EONs and through the methods of the present invention are present in the human CFTR gene, more preferably the splice mutations 621+1G>T and 1717-1G>A. The exact mutation does not have to be the target for the RNA editing; it may be that (for instance in the case of 621+1G>T) a neighbouring or nearby adenosine in the splice mutation is the target nucleotide, which conversion to I fixes the splice mutation back to a normal state. The skilled person is aware of methods to determine whether or not normal splicing is restored, after RNA editing of the adenosine within the splice mutation site or area.

An EON according to the present invention may be chemically modified almost in its entirety, for example by providing nucleotides with a 2′-O-methylated sugar moiety (2′-OMe) and/or with a 2′-O-methoxyethyl sugar moiety (2′-MOE). However, the nucleotide opposite the target adenosine does not comprise the 2′-OMe modification, and in yet a further preferred aspect, at least one and in a preferred aspect both the two neighbouring nucleotides flanking each nucleotide opposing the target adenosine further do not comprise a 2′-OMe modification. Complete modification, wherein all nucleotides within the EON holds a 2′-OMe modification results in a non-functional oligonucleotide as far as RNA editing goes, presumably because it hinders the ADAR activity at the targeted position. In general, an adenosine in a target RNA can be protected from editing by providing an opposing nucleotide with a 2′-OMe group, or by providing a guanine or adenine as opposing base, as these two nucleobases are also able to reduce editing of the opposing adenosine.

Various chemistries and modification are known in the field of oligonucleotides that can be readily used in accordance with the invention. The regular internucleosidic linkages between the nucleotides may be altered by mono- or di-thioation of the phosphodiester bonds to yield phosphorothioate esters or phosphorodithioate esters, respectively. Other modifications of the internucleosidic linkages are possible, including amidation and peptide linkers.

The ribose sugar may be modified by substitution of the 2′-O moiety with a lower alkyl (C1-4, such as 2′-O-Me), alkenyl (C2-4), alkynyl (C2-4), methoxyethyl (2′-MOE), or other substituent. Preferred substituents of the 2′ OH group are a methyl, methoxyethyl, F, constrained ethyl (cEt) or 3,3′-dimethylallyl group. The latter is known for its property to inhibit nuclease sensitivity due to its bulkiness, while improving efficiency of hybridization (Angus & Sproat FEBS 1993 Vol. 325, no. 1, 2, 123-7). Alternatively, locked nucleic acid sequences (LNAs), comprising a 2′-4′ intramolecular bridge (usually a methylene bridge between the 2′ oxygen and 4′ carbon) linkage inside the ribose ring, may be applied. Purine nucleobases and/or pyrimidine nucleobases may be modified to alter their properties, for example by amination or deamination of the heterocyclic rings. The exact chemistries and formats may depend from oligonucleotide construct to oligonucleotide construct and from application to application, and may be worked out in accordance with the wishes and preferences of those of skill in the art.

The EON according to the invention should normally be longer than 10 nucleotides, preferably more than 11, 12, 13, 14, 15, 16, still more preferably more than 17 nucleotides. In one embodiment the EON according to the invention is longer than 20 nucleotides. The oligonucleotide according to the invention is preferably shorter than 100 nucleotides, still more preferably shorter than 60 nucleotides. In one embodiment the EON according to the invention is shorter than 50 nucleotides. In a preferred aspect, the oligonucleotide according to the invention comprises 18 to 70 nucleotides, more preferably comprises 18 to 60 nucleotides, and even more preferably comprises 18 to 50 nucleotides. Hence, in a most preferred aspect, the oligonucleotide of the present invention comprises 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

It is known in the art, that RNA editing entities (such as human ADAR enzymes) edit dsRNA structures with varying specificity, depending on a number of factors. One important factor is the degree of complementarity of the two strands making up the dsRNA sequence. Perfect complementarity of the two strands usually causes the catalytic domain of hADAR to deaminate adenosines in a non-discriminative manner, reacting more or less with any adenosine it encounters. The specificity of hADAR1 and 2 can be increased by introducing chemical modifications and/or ensuring a number of mismatches in the dsRNA, which presumably help to position the dsRNA binding domains in a way that has not been clearly defined yet. Additionally, the deamination reaction itself can be enhanced by providing an EON that comprises a mismatch opposite the adenosine to be edited. The mismatch is preferably created by providing a targeting portion having a cytidine opposite the adenosine to be edited. As an alternative, also uridines may be used opposite the adenosine, which, understandably, will not result in a ‘mismatch’ because U and A pair. Upon deamination of the adenosine in the target strand, the target strand will obtain an inosine which, for most biochemical processes, is “read” by the cell's biochemical machinery as a G. Hence, after A to I conversion, the mismatch has been resolved, because I is perfectly capable of base pairing with the opposite C in the targeting portion of the oligonucleotide construct according to the invention. After the mismatch has been resolved due to editing, the substrate is released and the oligonucleotide construct-editing entity complex is released from the target RNA sequence, which then becomes available for downstream biochemical processes, such as splicing and translation. Also, this on/off rate is important because the targeting oligonucleotide should not be too tightly bound to the target RNA.

The desired level of specificity of editing the target RNA sequence may depend from target to target. Following the instructions in the present patent application, those of skill in the art will be capable of designing the complementary portion of the oligonucleotide according to their needs, and, with some trial and error, obtain the desired result.

The oligonucleotide of the invention will usually comprise the normal nucleotides A, G, U and C, but may also include inosine (I), for example instead of one or more G nucleotides.

To prevent undesired editing of adenosines in the target RNA sequence in the region of overlap with the oligonucleotide construct, the oligonucleotide may be chemically modified. It has been shown in the art, that 2′-O-methylation of the ribosyl-moiety of a nucleoside opposite an adenosine in the target RNA sequence dramatically reduces deamination of that adenosine by ADAR (Vogel et al. 2014). Hence, by including 2′-O-methyl (2′-OMe) nucleotides in desired position of the oligonucleotide construct, the specificity of editing is dramatically improved. Other 2′-O substitutions of the ribosyl moiety, such as 2′-O-methoxyethyl (2′-MOE) and 2′-O-dimethylallyl groups may also reduce unwanted editing of the corresponding (opposite) adenosine in the target RNA sequence. All these modifications may be applied in the oligonucleotides of the present invention. Other chemical modifications are also readily available to the person having ordinary skill in the art of oligonucleotide synthesis and design. The synthesis of such chemically modified oligonucleotides and testing them in methods according to the invention does not pose an undue burden and other modifications are encompassed by the present invention.

RNA editing molecules present in the cell will usually be proteinaceous in nature, such as the ADAR enzymes found in metazoans, including mammals. Preferably, the cellular editing entity is an enzyme, more preferably an adenosine deaminase or a cytidine deaminase, still more preferably an adenosine deaminase. These are enzymes with ADAR activity. The ones of most interest are the human ADARs, hADAR1 and hADAR2, including any isoforms thereof such as hADAR1 p110 and p150. RNA editing enzymes known in the art, for which oligonucleotide constructs according to the invention may conveniently be designed, include the adenosine deaminases acting on RNA (ADARs), such as hADAR1 and hADAR2 in humans or human cells and cytidine deaminases. Human ADAR3 (hADAR3) has been described in the prior art, but reportedly has no deaminase activity. It is known that hADAR1 exists in two isoforms; a long 150 kDa interferon inducible version and a shorter, 100 kDa version, that is produced through alternative splicing from a common pre-mRNA. Consequently, the level of the 150 kDa isoform present in the cell may be influenced by interferon, particularly interferon-gamma (IFN-gamma). hADAR1 is also inducible by TNF-alpha. This provides an opportunity to develop combination therapy, whereby interferon-gamma or TNF-alpha and oligonucleotides according to the invention are administered to a patient either as a combination product, or as separate products, either simultaneously or subsequently, in any order. Certain disease conditions may already coincide with increased IFN-gamma or TNF-alpha levels in certain tissues of a patient, creating further opportunities to make editing more specific for diseased tissues.

Examples of further chemical modifications in the EONs of the present invention are modifications of the sugar moiety, including by cross-linking substituents within the sugar (ribose) moiety (e.g. as in LNA or locked nucleic acids), by substitution of the 2′-O atom with alkyl (e.g. 2′-O-methyl), alkynyl (2′-O-alkynyl), alkenyl (2′-O-alkenyl), alkoxyalkyl (e.g. methoxyethyl, 2′-MOE) groups, having a length as specified above, and the like. In addition, the phosphodiester group of the backbone may be modified by alkylation, thioation, dithioation, amidation and the like to yield phosphonate, phosphorothioate, phosphorodithioate, phosphoramidate, etc., internucleosidic linkages. The internucleosidic linkages may be replaced in full or in part by peptidic linkages to yield in peptidonucleic acid sequences and the like. Alternatively, or in addition, the nucleobases may be modified by (de)amination, to yield inosine or 2′6′-diaminopurines and the like. A further modification may be methylation of the C5 in the cytidine moiety of the nucleotide, to reduce potential immunogenic properties known to be associated with CpG sequences.

In case the dsRNA complex recruits ADAR enzymes to deaminate an A to I in the target RNA sequence, the base-pair, mismatch, bulge or wobble between the adenosine to be edited and the opposite nucleotide may comprise an adenosine, a guanosine, a uridine or a cytidine, but preferably a cytidine residue. Except for the potential mismatch opposite the editing site (when no uridine is applied), the remaining portion of the EON may be perfectly complementary to the target RNA. However, as shown herein, in certain aspects the invention relates to EONs that comprise a limited number of imperfect matches. It will be understood by a person having ordinary skill in the art that the extent to which the editing entities inside the cell are redirected to other target sites may be regulated by varying the affinity of the oligonucleotides according to the invention for the recognition domain of the editing molecule. The exact modification may be determined through some trial and error and/or through computational methods based on structural interactions between the oligonucleotide and the recognition domain of the editing molecule.

In addition, or alternatively, the degree of recruiting and redirecting the editing entity resident in the cell may be regulated by the dosing and the dosing regimen of the oligonucleotide. This is something to be determined by the experimenter (in vitro) or the clinician, usually in phase I and/or II clinical trials.

The invention concerns the modification of target RNA sequences in eukaryotic, preferably metazoan, more preferably mammalian cells. In principle the invention can be used with cells from any mammalian species, but it is preferably used with a human cell. The invention can be used with cells from any organ e.g. skin, lung, heart, kidney, liver, pancreas, gut, muscle, gland, eye, brain, blood and the like. The invention is particularly suitable for modifying sequences in cells, tissues or organs implicated in a diseased state of a (human) subject, for instance when the human subject suffers from Cystic Fibrosis. Such cells include but are not limited to epithelial cells of the lung. The cell can be located in vitro or in vivo. One advantage of the invention is that it can be used with cells in situ in a living organism, but it can also be used with cells in culture. In some embodiments cells are treated ex vivo and are then introduced into a living organism (e.g. re-introduced into an organism from whom they were originally derived). The invention can also be used to edit target RNA sequences in cells within a so-called organoid. Organoids can be thought of as three-dimensional in vitro-derived tissues but are driven using specific conditions to generate individual, isolated tissues (e.g. see Lancaster & Knoblich, Science 2014, vol. 345 no. 6194 1247125). In a therapeutic setting they are useful because they can be derived in vitro from a patient's cells, and the organoids can then be re-introduced to the patient as autologous material which is less likely to be rejected than a normal transplant. The cell to be treated will generally have a genetic mutation. The mutation may be heterozygous or homozygous. The invention will typically be used to modify point mutations, such as N to A mutations, wherein N may be G, C, U (on the DNA level T), preferably G to A mutations, or N to C mutations, wherein N may be A, G, U (on the DNA level T), preferably U to C mutations.

Without wishing to be bound be theory, the RNA editing through hADAR1 and hADAR2 is thought to take place on primary transcripts in the nucleus, during transcription or splicing, or in the cytoplasm, where e.g. mature mRNA, miRNA or ncRNA can be edited. Different isoforms of the editing enzymes are known to localize differentially, e.g. with hADAR1 p110 found mostly in the nucleus, and hADAR1 p150 in the cytoplasm. The RNA editing by cytidine deaminases is thought to take place on the mRNA level.

The invention can be used to make a change in a target RNA sequence in a eukaryotic cell through the use of an oligonucleotide that is capable of targeting a site to be edited and recruiting RNA editing entities resident in the cell to bring about the editing reaction(s). Preferred editing reactions are adenosine deaminations, converting adenosines into inosines. The target RNA sequence may comprise a mutation that one may wish to correct or alter, such as a point mutation (a transition or a transversion). The target RNA may be any cellular or viral RNA sequence but is more usually a pre-mRNA or an mRNA with a protein coding function.

Many genetic diseases are caused by G to A mutations, and these are preferred target diseases because adenosine deamination at the mutated target adenosine will reverse the mutation to a codon giving rise to a functional, full length and/or wild type protein, especially when it concerns PTCs. Preferred examples of genetic diseases that can be prevented and/or treated with oligonucleotides according to the invention are any disease where the modification of one or more adenosines in a target RNA will bring about a (potentially) beneficial change. Especially preferred is Cystic Fibrosis, and more specifically the RNA editing of adenosines in the disease-inducing PTCs in CFTR RNA is preferred. Those skilled in the art of CF mutations recognise that between 1000 and 2000 mutations are known in the CFTR gene, including G542X, W1282X, R553X, R1162X, Y122X, W1089X, W846X, W401X, 621+1G>T or 1717-1G>A.

It should be clear, that targeted editing according to the invention can be applied to any adenosine (or cytosine), whether it is a mutated or a wild-type nucleotide in a given sequence. For example, editing may be used to create RNA sequences with different properties. Such properties may be coding properties (creating proteins with different sequences or length, leading to altered protein properties or functions), or binding properties (causing inhibition or over-expression of the RNA itself or a target or binding partner; entire expression pathways may be altered by recoding miRNAs or their cognate sequences on target RNAs). Protein function or localization may be changed at will, by functional domains or recognition motifs, including but not limited to signal sequences, targeting or localization signals, recognition sites for proteolytic cleavage or co- or post-translational modification, catalytic sites of enzymes, binding sites for binding partners, signals for degradation or activation and so on. These and other forms of RNA and protein “engineering”, whether or not to prevent, delay or treat disease or for any other purpose, in medicine or biotechnology, as diagnostic, prophylactic, therapeutic, research tool or otherwise, are encompassed by the present invention.

The target sequence is endogenous to the eukaryotic, preferably mammalian, more preferably human cell.

The amount of oligonucleotide to be administered, the dosage and the dosing regimen can vary from cell type to cell type, the disease to be treated, the target population, the mode of administration (e.g. systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change. It is possible that higher doses of oligonucleotide could compete for binding to a nucleic acid editing entity (e.g. ADAR) within a cell, thereby depleting the amount of the entity which is free to take part in RNA editing, but routine dosing trials will reveal any such effects for a given oligonucleotide and a given target.

One suitable trial technique involves delivering the oligonucleotide construct to cell lines, or a test organism and then taking biopsy samples at various time points thereafter. The sequence of the target RNA can be assessed in the biopsy sample and the proportion of cells having the modification can easily be followed. After this trial has been performed once then the knowledge can be retained and future delivery can be performed without needing to take biopsy samples. A method of the invention can thus include a step of identifying the presence of the desired change in the cell's target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively the change may be assessed on the level of the protein (length, glycosylation, function or the like), or by some functional read-out, such as a(n) (inducible) current, when the protein encoded by the target RNA sequence is an ion channel, for example. In the case of CFTR function, an Ussing chamber assay or an NPD test in a mammal, including humans, are well known to a person skilled in the art to assess restoration or gain of function.

After RNA editing has occurred in a cell, the modified RNA can become diluted over time, for example due to cell division, limited half-life of the edited RNAs, etc. Thus, in practical therapeutic terms a method of the invention may involve repeated delivery of an oligonucleotide construct until enough target RNAs have been modified to provide a tangible benefit to the patient and/or to maintain the benefits over time.

Oligonucleotides of the invention are particularly suitable for therapeutic use, and so the invention provides a pharmaceutical composition comprising an oligonucleotide of the invention and a pharmaceutically acceptable carrier. In some embodiments of the invention the pharmaceutically acceptable carrier can simply be a saline solution. This can usefully be isotonic or hypotonic, particularly for pulmonary delivery. The invention also provides a delivery device (e.g. syringe, inhaler, nebuliser) which includes a pharmaceutical composition of the invention.

The invention also provides an oligonucleotide of the invention for use in a method for making a change in a target RNA sequence in a mammalian, preferably human cell, as described herein. Similarly, the invention provides the use of an oligonucleotide construct of the invention in the manufacture of a medicament for making a change in a target RNA sequence in a mammalian, preferably human cell, as described herein.

The invention also relates to a method for the deamination of at least one specific target adenosine present in a target RNA sequence in a cell, the method comprising the steps of: providing the cell with an EON according to the invention; allowing uptake by the cell of the EON; allowing annealing of the EON to the target RNA sequence; allowing a mammalian ADAR enzyme comprising a natural dsRNA binding domain as found in the wild type enzyme to deaminate the target adenosine in the target RNA sequence to an inosine; and optionally identifying the presence of the inosine in the RNA sequence.

Introduction of the EON according to the present invention into the cell is performed by general methods known to the person skilled in the art. After deamination the read-out of the effect (e.g. alteration of the target RNA sequence) can be monitored through different ways. Hence, the identification step of whether the desired deamination of the target adenosine has indeed taken place depends generally on the position of the target adenosine in the target RNA sequence, and the effect that is incurred by the presence of the adenosine (point mutation, early stop codon). Hence, in a preferred aspect, depending on the ultimate deamination effect of A to I conversion, the identification step comprises: sequencing the target RNA; assessing the presence of a functional, elongated, full length and/or wild type protein; assessing whether splicing of the pre-mRNA was altered by the deamination; or using a functional read-out, wherein the target RNA after the deamination encodes a functional, full length, elongated and/or wild type protein. In the event that there is a UAA stop codon it means that both adenosines need to be deaminated. Hence, the invention also relates to oligonucleotides and methods wherein two adenosines that are next to each other are co-deaminated by an RNA editing enzyme such as ADAR. In this particular case, the UAA stop codon is converted into a UGG Trp-encoding codon. Because the deamination of the adenosine to an inosine may result in a protein that is no longer suffering from the mutated A at the target position, the identification of the deamination into inosine may also be a functional read-out, for instance an assessment on whether a functional protein is present, or even the assessment that a disease that is caused by the presence of the adenosine is (partly) reversed. The functional assessment for each of the diseases mentioned herein will generally be according to methods known to the skilled person. A very suitable manner to identify the presence of an inosine after deamination of the target adenosine is of course RT-PCR and sequencing, using methods that are well-known to the person skilled in the art.

The oligonucleotide according to the invention is suitably administrated in aqueous solution, e.g. saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be by inhalation (e.g. through nebulization), intranasally, orally, by injection or infusion, intravenously, subcutaneously, intra-dermally, intra-cranially, intravitreally, intramuscularly, intra-tracheally, intra-peritoneally, intra-rectally, and the like. Administration may be in solid form, in the form of a powder, a pill, or in any other form compatible with pharmaceutical use in humans. The invention is particularly suitable for treating genetic diseases, such as cystic fibrosis.

In some embodiments the oligonucleotide construct can be delivered systemically, but it is more typical to deliver an oligonucleotide to cells in which the target sequence's phenotype is seen. For instance, mutations in CFTR cause cystic fibrosis, which is primarily seen in lung epithelial tissue, so with a CFTR target sequence it is preferred to deliver the oligonucleotide construct specifically and directly to the lungs. This can be conveniently achieved by inhalation e.g. of a powder or aerosol, typically via the use of a nebuliser. Especially preferred are nebulizers that use a so-called vibrating mesh, including the PARI eFlow (Rapid) or the i-neb from Respironics. It is to be expected that inhaled delivery of oligonucleotide constructs according to the invention can also target these cells efficiently, which in the case of CFTR gene targeting could lead to amelioration of gastrointestinal symptoms also associated with cystic fibrosis. In some diseases the mucus layer shows an increased thickness, leading to a decreased absorption of medicines via the lung. One such a disease is chronical bronchitis; another example is cystic fibrosis. Various forms of mucus normalizers are available, such as DNases, hypertonic saline or mannitol, which is commercially available under the name of Bronchitol. When mucus normalizers are used in combination with RNA editing oligonucleotide constructs, such as the oligonucleotide constructs according to the invention, they might increase the effectiveness of those medicines. Accordingly, administration of an oligonucleotide construct according to the invention to a subject, preferably a human subject is preferably combined with mucus normalizers, preferably those mucus normalizers described herein. In addition, administration of the oligonucleotide constructs according to the invention can be combined with administration of small molecule for treatment of CF, such as potentiator compounds for example Kalydeco (ivacaftor; VX-770), or corrector compounds, for example VX-809 (lumacaftor) and/or VX-661. Other combination therapies in CF may comprise the use of an oligonucleotide construct according to the invention in combination with an inducer of adenosine deaminase, using IFN-gamma or TNF-alpha. Alternatively, or in combination with the mucus normalizers, delivery in mucus penetrating particles or nanoparticles can be applied for efficient delivery of RNA editing molecules to epithelial cells of for example lung and intestine. Accordingly, administration of an oligonucleotide construct according to the invention to a subject, preferably a human subject, preferably uses delivery in mucus penetrating particles or nanoparticles. Chronic and acute lung infections are often present in patients with diseases such as cystic fibrosis. Antibiotic treatments reduce bacterial infections and the symptoms of those such as mucus thickening and/or biofilm formation. The use of antibiotics in combination with oligonucleotide constructs according to the invention could increase effectiveness of the RNA editing due to easier access of the target cells for the oligonucleotide construct. Accordingly, administration of an oligonucleotide construct according to the invention to a subject, preferably a human subject, is preferably combined with antibiotic treatment to reduce bacterial infections and the symptoms of those such as mucus thickening and/or biofilm formation. The antibiotics can be administered systemically or locally or both. For application in cystic fibrosis patients the oligonucleotide constructs according to the invention, or packaged or complexed oligonucleotide constructs according to the invention may be combined with any mucus normalizer such as a DNase, mannitol, hypertonic saline and/or antibiotics and/or a small molecule for treatment of CF, such as potentiator compounds for example ivacaftor, or corrector compounds, for example lumacaftor and/or VX-661. To increase access to the target cells, Broncheo-Alveolar Lavage (BAL) could be applied to clean the lungs before administration of the oligonucleotide according to the invention.

### EXAMPLES

### Example 1: Design of Single-Stranded Antisense Editing Oligonucleotides with Phosphonoacetate Internucleotide Linkages Based on Computational Modelling

The inventors of the present invention envisioned that modelling data could possibly support the identification of structural features that could be incorporated into editing oligonucleotides (EONs) to improve (or to increase the efficiency of) editing of target RNA. The suboptimal molecular features were addressed by chemically modifying the nucleotides of the EONs so as to avoid steric hindrances with ADAR, to preserve protein-RNA intermolecular interactions observed in published structural data and even to provide a more efficient recruitment of the protein. To guide this process, the existing RNA-bound ADAR2 structures were used as a starting point (structural template). The published structure of double-stranded RNA in interaction with the ADAR2 deaminase domain (Matthews et al., Nature Structural and Molecular Biology, 2016) and a similar ADAR2 structure including a deaminase domain and double-stranded RNA binding domain (dsRBD) in interaction with a double-stranded RNA were analysed and a network of intra and intermolecular distances required for new structure calculations was generated. For the intra and intermolecular distance values, upper limits have been defined. For intramolecular distances, upper limits correspond to distances observed in the RNA-bound ADAR2 X-ray structure. For intermolecular distances, upper limits have been set between 1 to 3 Å above the observed distances allowing side-chain adaptation in the binding interface. For secondary structure elements of the ADAR2 deaminase domain and the double-stranded RNA binding domain, upper and lower distance limits were inserted to characterize the hydrogen-bond network classically detected in α-helices and β-sheets. Dihedral angle constraints have been derived from the published structure. This approach is based on standard methods used to solve protein-RNA structures in solution (Nuclear Magnetic Resonance Spectroscopy) that are known to the person skilled in the art, and integrates torsion angle as well as molecular dynamics steps. Calculated structures were generated of the ADAR2 deaminase domain connected by an artificial linker to the double-stranded RNA binding domain bound to functionally optimized EONs with CYANA3.97 (Herrmann et al., J.Mol.Biol., 2002) and we refined selected atomic models with the SANDER module of AMBER16 (Case D. A. et al., J. Comput. Chem., 2005) by simulated annealing in implicit water using the ff99SB force field. A representative atomic-scale model is shown in FIG. 5. In silico, a double-stranded RNA complex composed of EONs annealed to the Idua RNA target was used for illustration purposes. This protocol enabled the investigation of the atomic details of the interaction between the protein sidechains and the double-stranded RNA-EON helix. The interaction was modulated by chemical modifications of the oxygen-phosphate backbone of the EON.

Using this computational modelling approach, a series of atomic-scale model calculations was performed including punctual insertions of phosphonoacetate linkages (both oxygen-phosphate atoms simultaneously substituted) in a 32 nucleotide-long region embedding the deaminase domain and the dsRBD binding sites. For each linkage from the 3′ to the 5′ end of the EON, 200 RNA-bound deaminase domain and dsRBD ADAR2 structures were calculated and the 20 lowest energy ones were selected. In each of the 20 lowest energy structure, it was determined whether the relative position of the phosphonoacetate could prevent the formation of a potential hydrogen bond with the amino acids sidechains of the ADAR2 deaminase domain and dsRBD. Concomitantly, it was determined whether the relative position of the phosphonoacetate modification could generate steric clashes with the amino acid sidechains of the ADAR2 deaminase domain and its linked dsRBD (FIG. 2). In total 6400 structures were calculated, and the 640 most energetically favourable ones were screened for potential hydrogen bond contact disruptions and potential steric clashes. With this approach, relevant atomic scale pictures were obtained of the conformational space explored by phosphonoacetate modified linkages. Within the protein-EON binding interface, all positions prone to altering hydrogen bond formation between the EON sugar backbone and the surrounding protein sidechains were detected, as well as all positions likely to create steric clashes with the surrounding protein side-chains (FIG. 6). Following this novel modelling approach for therapeutic oligonucleotide design, it was concluded that specific positions within EONs bound to their RNA target do not tolerate phosphonoacetate linkages because of their propensity to disrupt, either significantly or moderately, the interaction with the ADAR2 deaminase and double-stranded RNA binding domains. The mapping of tolerability of phosphonoacetate linkages is set out in FIG. 3.

### Example 2: Design of Single-Stranded Antisense Editing Oligonucleotides with Unlocked Nucleic Acid Ribose Modifications Based on Computational Modeling

The computational modeling approach set out in Example 1 was also used to investigate nucleic acid ribose modifications. In this context, a series of structural calculations was performed including punctual insertions of unlocked nucleic acids in a 32 nucleotide-long region embedding the deaminase domain and the dsRBD binding sites. For each nucleotide from the 3′ to the 5′ end of the EON, 200 RNA-bound deaminase domain ADAR2 structures were calculated and the 20 lowest energy ones were selected. In each of the 20 lowest energy structure, it was determined whether the relative position of the unlocked nucleic acid modification could prevent the formation of a potential hydrogen bond with the amino acid sidechains of the ADAR2 deaminase domain and dsRBD. In total 6400 structures were calculated, and the 640 most energetically favourable ones were screened for potential intermolecular hydrogen bond contact disruptions. With this approach, relevant atomic scale pictures were obtained of the conformational space explored by the unlocked nucleic acids. Within the protein-EON binding interface, all positions prone to altering hydrogen bond formation between the EON sugar backbone and the surrounding protein sidechains were detected (as shown, for example, in FIG. 7). The tolerability regarding the insertion of unlocked nucleic acids is determined in a position-based analysis. UNAs are responsible for local dynamic changes and two or more consecutive unlocked nucleic acids may drastically affect the protein-RNA interactions network. With two or more consecutive UNAs, the effects on binding compatibility are expected to differ significantly from the results of the position-based computational approach. Following this novel modelling approach for therapeutic oligonucleotide design, it was concluded that specific positions within EONs disrupt binding with ADAR2, either significantly or moderately, with unlocked nucleic acids because of their propensity to alter the interaction with the ADAR2 deaminase and double-stranded RNA binding domains. The mapping of tolerability of unlocked nucleic acid ribose modifications is set out in FIG. 4.

### Example 3: Editing of a Non-Sense Mutation in GFP Target RNA Using an EON Containing UNA Nucleotides

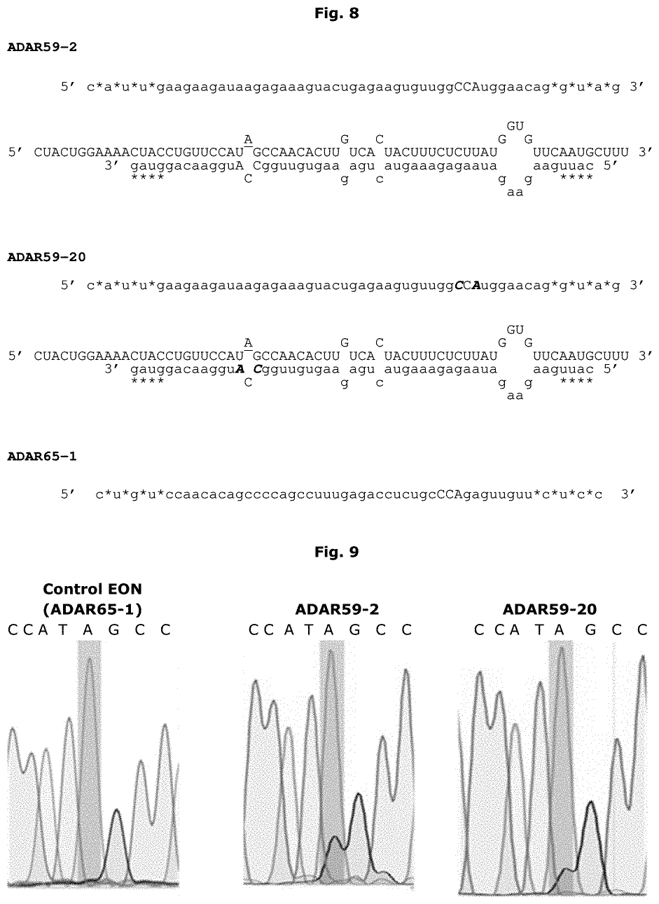

The effect of UNA modifications on RNA editing was investigated in a cell system using HeLa cells that contain an expression construct encoding a Green Fluorescent Protein (GFP), stably integrated into the cellular genome. In this construct, a stop codon (TAG) has been introduced at codon position 57, resulting in a triplet UAG in the mRNA. Editing of the A within the UAG triplet would result in a UIG (functionally UGG), representing a Trp codon, and subsequently functional GFP protein. It was investigated whether the middle A in this triplet could be deaminated to an I (which would subsequently be read as a G), using an EON that contained to UNA residues that base-pair to the nucleotides on either side of the target A (FIG. 8).

It was investigated whether sequence analysis would reveal that an EON with such modifications could edit the adenosine at that position, similar to a positive control with only RNA in the region complementary to the target triplet. For this, 0.3×10⁶ HeLa cells stably expressing the GFPstop57 construct were seeded per well (6-well plates) in Dulbecco's modified Eagle's medium with 10% fetal bovine serum. After 24 h, cells for subsequent AON transfections were transfected with 1 μg ADAR2 overexpression plasmid (RC209073; Origene) using Lipofectamine 2000. The following day, cell samples were transfected with 100 nM of each AON (see FIG. 8) using Lipofectamine 2000. After another 24 h, RNA was isolated from lysed cells and used as a template for cDNA synthesis. Analysis of RNA editing was performed by RT-PCR (forward primer 5′-AGAGGGTGAAGGTGATGCAA-3′ (SEQ ID NO: 2) and reverse primer 5′-GGGCATGGCACTCTTGAAAA-3′ (SEQ ID NO: 3), followed by Sanger sequencing of the PCR product, using general RT-PCR and sequencing methods known to the person skilled in the art.

Efficiency of A-to-I editing was analyzed by Sanger sequencing of the RT-PCR products, where A-to-I editing should be apparent in the sequencing chromatogram as (a partial) shift in the intensity of the signal from A to G (FIG. 9). While the method is not fully quantitative, the ratio of the A and G frequencies can be used as an approximate estimation of the A-to-I editing efficiency. As expected, no signal for G is observed overlapping the A peak at the target site in samples that were transfected with a control EON of an unrelated sequence (ADAR65-1). In contrast, in samples transfected with a positive control EON (ADAR59-2) a partial change into a G is observed at this position, as indicated by the overlapping A and G peaks (green and black in FIG. 9, respectively). Similarly, the sample transfected with the EON containing the two UNA residues also showed, a partial change into a G is observed at the target position, indicating that the replacement of RNA nucleotides in these positions with UNA is compatible with ADAR2-mediated A-to-I editing.

1. An editing oligonucleotide (EON) capable of forming a double stranded complex with a target nucleic acid molecule in a cell, and capable of recruiting an enzyme with nucleotide deaminase activity, wherein the target nucleic acid molecule comprises a target nucleotide for deamination by the enzyme with nucleotide deamination activity, wherein the EON comprises a nucleotide, referred to as nucleotide position 0, which is opposite the target nucleotide and which forms a mismatch with the target nucleotide, and wherein the internucleotide linkage numbering is such that linkage number 0 is the linkage 5′ from nucleotide position 0, and wherein the nucleotide positions and the linkage positions in the EON are both positively (+) and negatively (−) incremented towards the 5′ and 3′ ends, respectively, characterized in that (i) the EON comprises at least one phosphonoacetate internucleotide linkage and at least one internucleotide linkage that is not a phosphonoacetate internucleotide linkage, and/or (ii) the EON comprises at least one nucleotide comprising an unlocked nucleic acid (UNA) ribose modification and at least one nucleotide not comprising a UNA ribose modification.

2. The EON according to claim 1, wherein the at least one phosphonoacetate internucleotide linkage is at linkage position +19, +18, +17, +16, +15, +14, +10, +9, +5, +4, +3, +2, +1, 0, −6, −7, −8, −9, −10, −11 and/or −12.

3. The EON according to any preceding claim, wherein there is not a phosphonoacetate internucleotide linkage at linkage position +13, +12, +11, +8, +7, +6, −1, −2, −3, −4 and/or −5.

4. The EON according to any preceding claim, wherein the internucleotide linkages that are not phosphonoacetate internucleotide linkages are internucleotide linkages independently selected from phosphorothioate, phosphodithioate, 3′-methylenephosphonate, 5′-methylenephosphonate, and/or 3′-phosphoroamidate.

5. The EON according to any preceding claim, wherein the EON comprises a UNA ribose modification at position +19, +18, +17, +16, +15, +11, +10, +9, +8, +7, +6, +5, +4, +3, +1, −1, −2, −4, −5, −6, −7, −8, −9, −10, −11 and/or −12.

6. The EON according to any preceding claim, wherein the EON does not comprise a UNA ribose modification at position +14, +13, +12, +2, 0, and/or −3, preferably wherein the EON does not comprise a UNA ribose modification at position 0.

7. The EON according to any preceding claim, wherein the EON does not comprise UNA ribose modifications at consecutive positions.

8. The EON according to any preceding claim, wherein the EON comprises both (i) at least one phosphonoacetate internucleotide linkage, and (ii) at least one nucleotide comprising an unlocked nucleic acid (UNA) ribose modification.

9. The EON according to any preceding claim, further comprising one or more nucleotides comprising a 2′-O-methoxyethyl (2′-MOE) ribose modification, wherein the EON comprises one or more nucleotides not comprising a 2′-MOE ribose modification.

10. The EON according to any preceding claim, wherein the EON comprises 2′-O-methyl (2′-OMe) ribose modifications or deoxynucleotides at positions other than position 0 that do not comprise a 2′-MOE ribose modification.

11. The EON according to any preceding claim, wherein the enzyme with nucleotide deaminase activity comprises a deaminase domain with adenosine deamination activity, preferably ADAR1 or ADAR2, more preferably ADAR2.

12. The EON according to any preceding claim, wherein the enzyme with nucleotide deaminase activity is a naturally expressed eukaryotic adenosine deamination enzyme.

13. The EON according to any preceding claim, wherein the EON is at least 10, 11, 12, 13, 14, 15, 16, 17, 18, 19, 20, 21, 22, 23, 24, 25, 26, 27, 28, 29, 30, 31, or 32 nucleotides in length, and wherein the EON is shorter than 100 nucleotides, preferably shorter than 60 nucleotides.

14. The EON according to any preceding claim, wherein the target nucleotide is an adenosine that is deaminated to an inosine.

15. The EON according to claim 14, wherein the adenosine is located in a UGA or UAG stop codon.

16. A pharmaceutical composition comprising the EON as characterized in any one of claims 1 to 15, and a pharmaceutically acceptable carrier.

17. The EON according to any of claims 1 to 15, or the pharmaceutical composition according to claim 16, for use in the treatment or prevention of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, ß-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, Sturge-Weber Syndrome, and cancer.

18. Use of the EON according to any one of claims 1 to 15, or the pharmaceutical composition according to claim 16, in the manufacture of a medicament for the treatment or prevention of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, ß-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, Sturge-Weber Syndrome, and cancer.

19. A method for the deamination of at least one target nucleotide present in a target nucleic acid molecule in a cell, the method comprising the steps of:
(i) providing the cell with an EON according to any one of claims 1 to 15, or the pharmaceutical composition according to claim 16;
(ii) allowing uptake by the cell of the EON;
(iii) allowing annealing of the EON to the target nucleic acid molecule;
(iv) allowing a mammalian enzyme with nucleotide deaminase activity to deaminate the target nucleotide in the target nucleic acid molecule; and
(v) optionally identifying the presence of the deaminated nucleotide in the target nucleic acid molecule.

20. The method of claim 19, wherein step (v) comprises:
a) sequencing a region of the target nucleic acid molecule, wherein said region comprises the deaminated target nucleotide;
b) assessing the presence of a functional, elongated, full length and/or wild type protein when the target nucleotide is an adenosine located in a UGA or UAG stop codon, which is edited to a UGG codon through the deamination;
c) assessing the presence of a functional, elongated, full length and/or wild type protein when two target adenosines are located in a UAA stop codon, which is edited to a UGG codon through the deamination of both target adenosines;
d) assessing, when the target nucleic acid is pre-mRNA, whether splicing of the pre-mRNA was altered by the deamination; or
e) using a functional read-out, wherein the target nucleic acid after the deamination encodes a functional, full length, elongated and/or wild type protein.


SEQUENCE LISTING (7 sequences)

SEQ ID NO: 1 - RNA, 32 nt, Artificial Sequence
  antisense RNA
agccuuugagaccucugcccagaguuguucuc

SEQ ID NO: 2 - DNA, 20 bp, Artificial Sequence
  primer
agagggtgaaggtgatgcaa

SEQ ID NO: 3 - DNA, 20 bp, Artificial Sequence
  primer
gggcatggcactcttgaaaa

SEQ ID NO: 4 - RNA, 53 nt, Artificial Sequence
  oligonucleotide construct
cauugaagaagauaagagaaaguacugagaaguguuggccauggaacagguag

SEQ ID NO: 5 - RNA, 53 nt, Artificial Sequence; identical to SEQ ID NO: 4
  oligonucleotide construct

SEQ ID NO: 6 - RNA, 68 nt, Artificial Sequence
  target construct
cuacuggaaaacuaccuguuccauagccaacacuugucacuacuuucucuuaugguguucaaugcuuu

SEQ ID NO: 7 - RNA, 50 nt, Artificial Sequence
  oligonucleotide construct
cuguccaacacagccccagccuuugagaccucugcccagaguuguucucc